(19) United States
(12) Patent Application Publication
(10) Pub. No.: US 2006/0161007 A1
(43) Pub. Date: Jul. 20, 2006

(54) Oxazole and thiazole combinatorial libraries

(76) Inventors: Lenore M. Martin, North Kingstown, RI (US); Bi-Huang Hu, Chicago, IL (US)

Correspondence Address: Richard L. Stevens, Esq.;Samuels, Gauthier & Stevens LLP, Suite 3300, 225 Franklin Street, Boston, MA, 02110, US

(21) Appl. No.: 11/266,046

(22) Filed: Nov. 3, 2005

Related U.S. Application Data
(63) Continuation of application No. 09/936,972, filed on Jan. 23, 2002, now abandoned.
(60) Provisional application No. 60/125,501, filed on Mar. 22, 1999.

(51) Int. Cl. C07D 277/56

(52) U.S. Cl. 548/200

(57) ABSTRACT

This invention provides a novel method for synthesizing an ensemble of peptides that allows for the generation of an unlimited number of antibiotic compounds. More specifically, the method comprises utilizes synthetic heterocyclic amino acids containing thaizole and/or oxazole as building blocks in a solid phase combinatorial synthesis to yield natural and unnatural antibiotic compounds.

### BACKGROUND OF THE INVENTION

1. Field of the Invention

This invention relates to the syntheses of thiazole and/or oxazole-containing amino acids and more specifically to the use of those compounds in a combinatorial synthesis to generate antibiotic compounds.

2. Description of the Related Art

More and more thiazole and/or oxazole-containing peptides with important biological activities such as antitumor, antifungal, antibiotic, and antiviral activities have been found from microbial and marine origins. It seems that the thiazole and oxazole ring systems might be important pharmacophores in those biologically active compounds.

Bleomycin A₂ is a clinically used antitumor drug. Antibiotic GE 2270A is a novel inhibitor of bacterial protein synthesis. Antibiotic A 10255 factor B is a bacteriocide. Trioxazole-containing macrolides ulapualides, kabiramides, halichondramides, myalolides and jaspisaides show antifungal activity. Moreover, ulapualides inhibit L1020 leukemia cell proliferation and halichondramides inhibit cell division. Tantazole A is a member of a unique family of mirabazoles and tantazoles which show selective toxicity against solid tumors, and thiangazole is a novel inhibitor of HIV-1. BE 10988, a potent inhibitor of topoisomerase II, inhibited the relaxation of pBP,322 plasmid DNA by topoisomerase II and significantly inhibited the growth of adriamycin and vincristine resistant P-388 murine leukemia as well as sensitive P-388 cell line.

Microcin B17, a peptide antibiotic with four thiazole and four oxazole rings. induces double-strand cleavage of DNA in a DNA gyrase-dependent reaction.

More interestingly, Escherichia coli sbmA mutants, which lack the inner membrane protein (SbmA) involved in microcin B17 uptake, were found to be resistant to bleomycin.

The traditional synthesis of biologically active compounds, such as compounds comprised of thiazole and/or oxazole compounds,. has involved the optimization of a lead compound. usually derived from biological sources. The optimization process through traditional synthesis, purification, characterization and screening is lengthy, painstaking and expensive. With the need to find more efficient methods of drug discovery and the advances in molecular biology and gene technology resulting in “high-throughtput screening”, combinatorial synthesis represents a new method to simultaneously generate many different compounds with defined structures to accelerate the search for new lead compounds and their optimization (including their structure-activity relation).

Combinatorial synthesis can be performed either in solution or on solid phase. Solid phase synthesis was introduced by R. B. Merrifield in an effort to overcome many problems of peptide synthesis in solution. In 1963, Merrifield published the first solid phase synthesis of a tetrapeptide in Merrifield, Solid Phase Synthesis Peptide Synthesis: The Synthesis of a Tetrapeptide, Journal of the American Chemical Society 85, 2149-2154 (1963). Today, the development of solid phase synthesis has extended to the syntheses of other biopolymers such as polynucleotides and polysaccharides, recently to the synthesis of small organic compounds and combinatorial synthesis.

Solid phase peptide synthesis is based on the attachment of α-amino and side-chain protected amino acid residues to an insoluble polymeric support (usually resin in peptide synthesis), followed by stepwise addition of protected amino acids to assemble the peptide chain on the solid support. After the attachment of the first α-amino and side-chain protected amino acid residue to the resin and the removal of the α-amino protecting group, a second α-amino and side-chain protected amino acid residue is attached to the free amino group of the resin-bound amino acid through the formation of an amide bond under the activation of a coupling reagent. Through this cycle, a planned peptide sequence can be assembled on the resin. Finally, the synthesized peptide chain can be cleaved from the resin and the side-chain protecting groups on the amino acid residues were removed simultaneously to obtain the expected peptide.

The present invention provides a novel method for the production of biologically active compounds comprised of thiazole and/or oxazole ring systems which overcomes the limitations associated with the traditional syntheses of biologically active compounds comprised of thiazole and/or oxazole ring systems. Moreover, the present invention provides a large array of diverse compounds comprised of thiazole and/or oxazole ring systems which can be screened for biological activity, and as described below, are biologically active.

### SUMMARY OF THE INVENTION

Broadly this invention is directed toward a novel method for synthesizing an ensemble of peptides that allows for the generation of an unlimited number of antibiotic compounds. The compounds synthesized find utility in inhibiting DNA replication or DNA transcription in cancer cells, pathogenic cells such as bacteria, and virally infected cells. The invention utilizes synthetic unnatural heterocyclic amino acids as building blocks in a solid phase combinatorial synthesis. More specifically, this invention is directed toward combining synthetic heterocyclic amino acids containing thiazole and/ or oxazole as building blocks in the synthesis of combinatorial libraries.

In a preferred embodiment of the invention, N-protected thiazole and/or oxazole containing amino acids are synthesized. These compounds are set forth below:

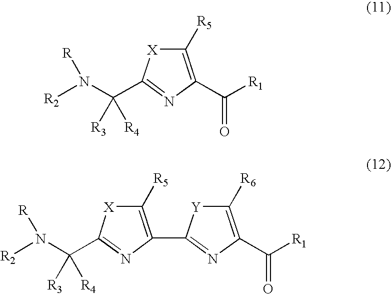

where R═H, a naturally occurring or synthetic L or D amino acid, Tert-butyloxycarbonyl (Boc), 9-fluorenylmethoxycarbonyl (Fmoc), carbobenzoxy (Z), Benzozyl (Bz), and other like amino protecting groups;

where R₁═OH, alkyl esters, aromatic esters such as methyl, ethyl, t-butyl and benzyl, activated esters such as pentafluorophenyl, nitrophenyl, N-hydroxysuccinimide, acid chlorides, fluorides, organic salts, such as cyclohexyamines (CHA), amides, an amide bonded to a linker such as a diamine, or an insoluble support for use in solid phase synthesis;

where R₂═H, a C₁-C₁₀ alkyl or an aromatic ring;

where R₃₋₄═H, or a C₁-C₁₀ alkyl;

where R₅₋₆═H, C₁-C₁₀ alkyl, a heterocylic ring, an aliphatic or aromatic ring, a functional group such as an amine, an alchohol, a halide or an organometallic complex;

where X═oxygen (O) or sulfur (S);

where Y═oxygen (O) or sulfur (S);

The building blocks 11 and 12 are coupled with natural amino acids in a solid phase combinatorial synthesis to yield libraries of antibiotic compounds.

One aspect of the invention is the syntheses which form compounds 11 and 12.

Another aspect of the invention is compound 12 where X═O and Y═S.

Another aspect of the invention is the coupling of compounds 11 and 12 with natural amino acids to yield naturally occurring antibiotic compounds.

Still another aspect of the invention are the antibiotic compounds that form the libraries.

Still another aspect of the invention are the syntheses which form the antibiotic compounds.

Still another embodiment of the invention is the solid phase combinatorial 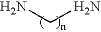
synthesis where a distinct linker molecule having the structure:

where n=1-10

is used to attach a building block to a solid support.

Still another embodiment of the invention is the combination of the solid phase-linker-building block(s).

The advantages of the invention are that the synthesized building blocks, 11 and 12 have restricted conformations that are presented in synthetic packages (Fmoc or Boc) which facilitates their incorporation into standard peptide methodology. Another advantage of the invention is that the design of the synthesis for the building blocks is flexible enough to allow the preparation of any combination of oxazole and thiazole rings in a given two-ring building block, such as compound 12, where X═O and Y═S, from naturally occurring amino acid starting materials. Furthermore, the peptide library can also incorporate any commercially available amino acid without the development of new chemistry.

Another aspect of the invention embodies the libraries of antibiotic compounds formed by the coupling at least one of the following compounds:

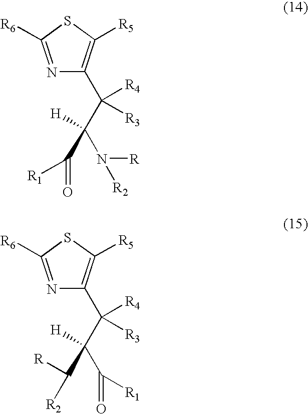

where R═H, a naturally occurring or synthetic L or D amino acid, Tert-butyloxycarbonyl (Boc), 9-fluorenylmethoxycarbonyl (Fmoc), carbobenzoxy (Z), Benzozyl (Bz), and other like amino protecting groups;

where R₁═OH, alkyl esters, aromatic esters such as methyl, ethyl, t-butyl and benzyl, activated esters such as pentafluorophenyl, nitrophenyl, N-hydroxysuccinimide, acid chlorides, fluorides, organic salts, such as cyclohexyamines (CHA), amides, an amide bonded to a linker such as a diamine, or an insoluble support for use in solid phase synthesis;

where R₂═H, a C₁-C₁₀ alkyl, or an aromatic ring;

where R₃₋₄═H, or a C₁-C₁₀ alkyl;

where R₅₋₆═H, C₁-C₁₀ alkyl, a heterocylic ring, an aliphatic or aromatic ring, a functional group such as an amine, an alchohol, a halide or an organometallic complex;

with natural amino acids in a solid phase combinatorial synthesis to yield libraries of antibiotic compounds.

In the above structures the stereochemistry of the chiral R groups can independently be in the R or S configuration or a mixture of the two.

### BRIEF DESCRIPTION OF THE DRAWING(S)

FIG. 1 is a schematic showing one embodiment of the novel synthesis of compound 2-(Fmoc-aminomethyl)-thiazole-4-carboxylic acid.

FIG. 2 is a schematic showing one embodiment of the novel synthesis of 2-(Fmoc-aminomethyl)-oxazole4-carboxlic acid.

FIG. 3 is a schematic showing one embodiment of the novel synthesis of 2-(2′-Fmoc-aminomethyl-oxazole-4′-yl)-thiazole-4-carboxylic acid.

FIG. 4 is a graph showing the effects of L2-6 and L2-9 on the growth of marine bacterium Vibrio angullarum.

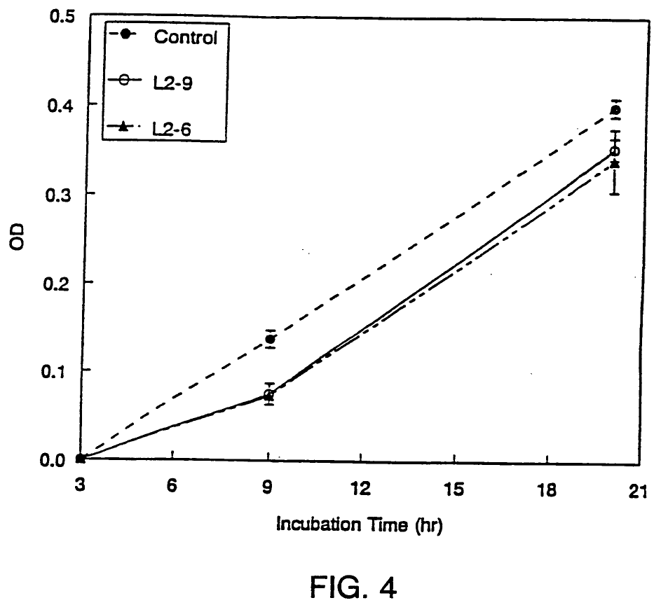

FIG. 5 is a graph showing the effect of a Microcin B17 fragment synthesized according to one embodiment of the invention on the growth of marine bacterium Vibrio angullarum.

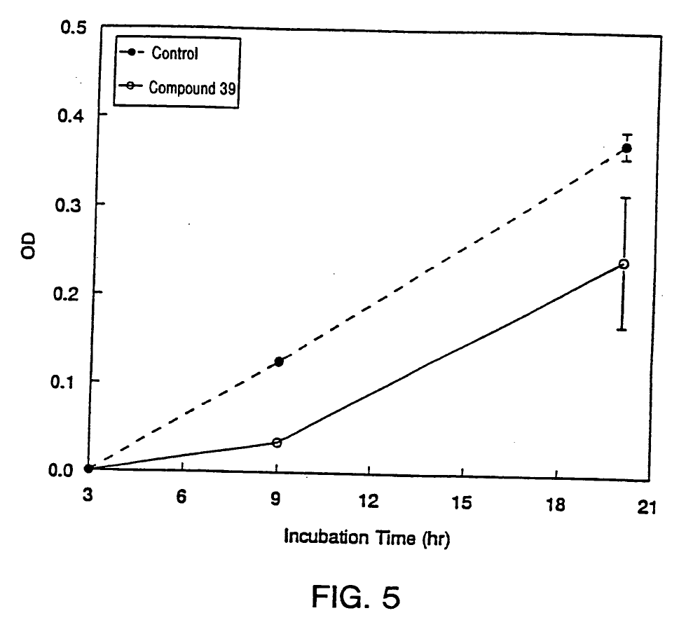

FIG. 6 is a graph showing the effect of peptide control tachyplesin on the growth of marine bacterium Vibrio angullarum.

### DESCRIPTION OF THE PREFERRED EMBODIMENT(S)

Results and Discussion

Synthesis of 2-(Fmoc-aminomethyl)-thiazole4-carboxylic Acid (1)

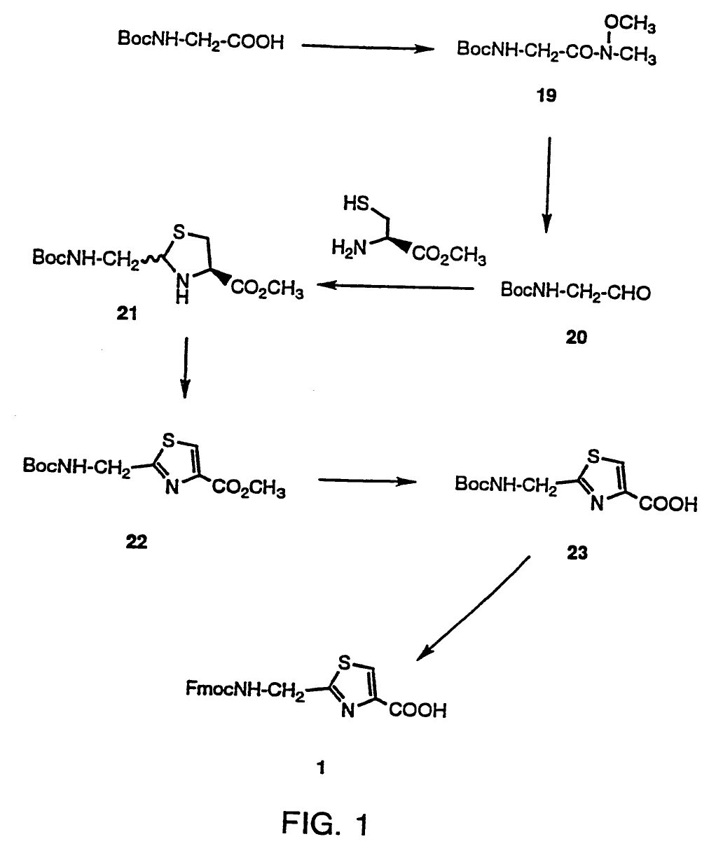

The preparation of compound 1 and R₂₋₆═H, using the Hantzsch synthesis has been reported. Referring to FIG. 1, the synthetic strategy disclosed herein is totally different from the reported one.

Cyclocondensation of the Boc-amino aldehyde prepared from its Boc-amino acid via the N-methoxy-N-methyl amide with L-cysteine methyl ester provided the thiazolidine, followed by dehydrogenation with active manganese dioxide to afford the thiazole product.

The coupling between Boc-glycine and O,N-imethylhydroxylamine hydrochloride with benzotriazole-1-yl-oxy-tris-(dimethylamino)-phosphonium hexafluorophosphate (BOP) and with 10-min. preactivation in the presence of N, N-diisopropylethylamine (DIEA) (31) at room temperature in 20 min. afforded the amide 19. This reaction was fast and proceeded cleanly with a high yield (80%). The characteristic signals in the ¹H-NMR spectrum of 19 at δ 3.70 (s, 3 H, —HN—OCH₃), 3.18 (s, 3 H, —NH—CH₃), and 1.45 (s, 9 H, t-butyl-O—) confirmed the formation of 19. 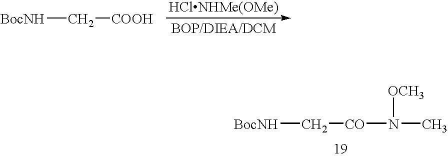
N-methoxy-N-methylamides are well known in the art as carbonyl equivalents in organic synthesis. The advantages of the use of this synthesis is the ease of preparation, and selective reduction to form the aldehydes. N-methoxy-N-methylamides can be prepared from the corresponding carboxylic acids and N, O-dimethylhydroxylamine with peptide coupling reagents such as BOP, DCC and i-butyl chloroformate.

The prepared Boc-Gly-N-methoxy-N-methylamide 19 in anhydrous THF was reduced with lithium aluminum anhydride in anhydrous diethyl ether for 30 min. at 0° C., followed by addition of a solution of potassium hydrogensulfate to afford 20 in high is yield (89%). The ¹H-NMR spectrum showed the expected signal of the aldehyde proton at δ 9.60 (s, 1 H). In the product, a small amount of impurities were detectable on TLC (hexane-EtOAc=1:1). 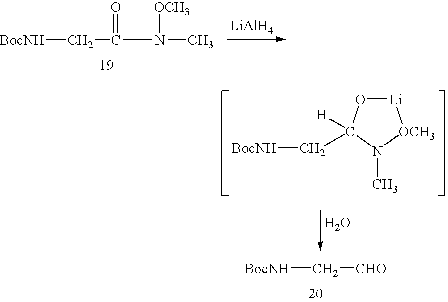
Reduction of 19 with lithium aluminum hydride gave a stable complex which prevented further reduction to the alcohol. Upon hydrolysis of the complex, the expected aldehyde was formed. If the solubility of the N-methoxy-N-methylamide is low in ethyl ether, then the N-methoxy-N-methylamide can be reduced in anhydrous THF. When TBF was used, it was found that a mixture of ether/THF achieved higher yield than did THF alone, and lower percentages of THF in ether gave higher yields.

The cyclcondensation of Boc-glycinal 20 with L-cysteine methyl ester was achieved by dropwise addition of a solution of L-cysteine methyl ester hydrochloride and DIEA in methylene chloride to a solution of 20 in methylene chloride at room temperature. The reaction instantly afforded 21 (77%). The ¹H-NMR spectrum showed that 21 is a mixture of the two possible diastereomeric thiazolidines (car 50:50). 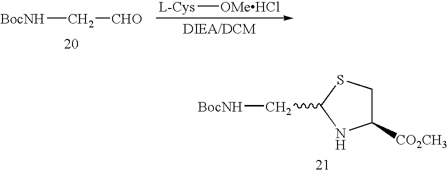
Unlike the conditions reported in the literature this condensation reaction was stirred overnight in benzene or in a slurry of magnesium sulfate in methylene chloride, it was found this reaction finished smoothly and instantly in methylene chloride. There is no need to prolong this reaction overnight or add magnesium sulfate to the reaction solution.

The dehydrogenation of 2-Boc-aminomethyl-thiazolidine-4-carboxylic methyl ester 21 was performed in benzene with manganese (IV) oxide (activated) at 55° C. for 60 min to afford 22 (60%). The ¹H-NMR spectrum showed the expected signal of aromatic proton at δ 8. 10 (s, 1 H). The UV spectrum displayed a maximum absorbance at 236 nm which is consistent with the reported data for thiazole rings. 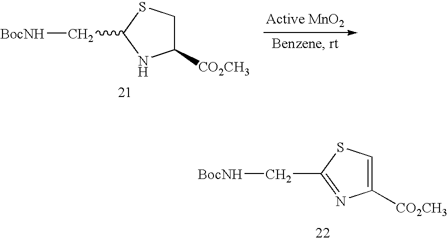
Dehydrogenation on active manganese dioxide can proceed either by an ionic mechanism or a free radical mechanism. The precise elucidation of the mechanism is difficult because of the nature of the heterogeneous reaction involved. A large excess of active manganese dioxide (ca. 30 eq.) was required for the efficient dehydrogenation of 21. The purity of 21 played a critical role in the success or failure of this reaction. It was found that the oxidation of crude 21 by active manganese dioxide produced a complicated product mixture (dark solution and many spots on the TLC), resulting in a low yield (ca. 10%). With a large excess of active manganese dioxide and purified 22, this reaction finished smoothly and cleanly within 60 min.

Alkaline hydrolysis of 22 in a THF/water solution (5:1) afforded 2-Boc-aminomethyl-thiazole4-carboxylic acid 23 in high yield (92%).

The conversion of the Boc protecting group of 23 to the Fmoc protecting group was achieved by removal of Boc protecting group of 23 with ThA in methylene chloride (1:1), followed by reaction with Fmoc-OSu to re-protect the amino group of 23 with Fmoc to provide 2-(Fmoc-aminomethyl)-thiazole4-carboxylic acid 1. RP-HPLC analysis showed that the product has one peak. The ESI-MS measured molecular weight of 1 is consistent with the calculated mass.

After the Boc deprotection of 23, the residue was neutralized with sodium carbonate and used without purification in the next step. Protection of the free amino group from 23 with Fmoc-OSu was achieved by reacting the compounds in a THF/water (2:1) solution in the presence of sodium carbonate (1 eq.). Unlike the normal preparation of Fmoc-amino acids, an excess of Fmoc-OSu was used because the unusual amino acid is more expensive. It was hard to remove Fmoc-OSu from the product by recrystallization. This, after the reaction was finished, washing the reaction mixture with methylene chloride was a necessary and simple way to remove the excess reagent.

Synthesis of 2-(Fmoc-aminomethyl)-oxazole-4-carboxylic acid (2)

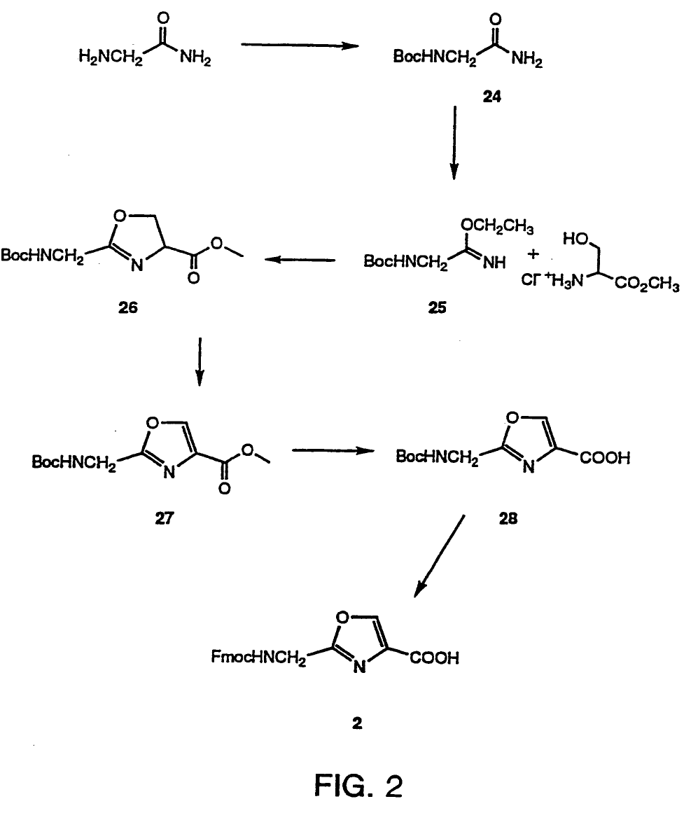

2-Fmoc-aminomethyl-oxazole4-carboxylic acid (2), where R=Fmoc, R₁═OH, and R₂₋₆═H, was synthesized before by the amino ether method. Referring to FIG. 2., we used the same strategy as reported: cyclocondensation of Boc amino acid amino ether and L-serine methyl ester hydrochloride salt afforded the oxazoline, followed by dehydrogenation to produce the corresponding oxazole amino acid. The difference the method disclosed herein and the reported one is that triethyloxonium tetrafluoroborate replaced triethyloxonium hexafluoro-phosphate in the amino ether preparation step and the CuBr₂/DBU/HMTA reagent was used instead of the DBU/CCl₄/acetonotrile/pyridine reagent in the dehydrogenation step.

Boc-Glycine amide (24) was prepared according to the method reported in Stewart et al., Solid Phase Peptide Synthesis, 2^{nd} ed.. 63, Pierce Chemical Compnay, Illinois, (1984), which is hereby incorporated by reference in its entirety into this disclosure. Di-t-butyl dicarbonate was added dropwise to a solution of glycine amide hydrochloride and one equivalent of sodium hydroxide in a water/t-butanol mixture (1:2) over a period of 15 min. After 15 min, more t-butanol was added to the reaction solution. The reaction was smooth and fast, finishing within one hour (yield 82%). The ¹H-NMR spectrum showed the signal of Boc at δ 1.46 (s, 9 H, t-butyl-O—), and confirmed the formation of 24. 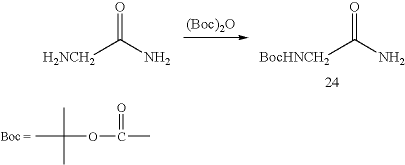
Amide 24 can easily dissolve in either methylene chloride or water. Therefore, after the reaction is finished and the t-butanol is removed, the volume of the residual aqueous mixture should be kept to a minimum. Otherwise, organic solvents such as ethyl acetate were unable to efficiently extract 24 from large volumes of aqueous mixtures for the purpose of purification. We found that DCM-benzene was a good system for recrystallizing amide 24.

To prepare Boc-aminoacetimino ethyl ether (25), Boc-glycine amide (24) was dissolved in a large volume of methylene chloride under argon, and was treated with triethyloxonium tetrafluoroborate for six hours at room temperature. Then, the reaction solution was diluted with more methylene chloride and the mixture was neutralized by pouring it into an icy sodium bicarbonate solution to afford 25. The ¹H-NMR spectrum displayed the signals of an ethyl ether at δ 4.16 (q, 2 H, J=7.0 Hz, —O—CH₂—CH₃) and 1.29 (t, 3 H, J=7.0 Hz, —O—CH₂—CH₃), and the Boc at 1.46 (s, 9 H, t-butyl-O-), consistent with the structure of Boc-aminoacetimino ethyl ether 25. 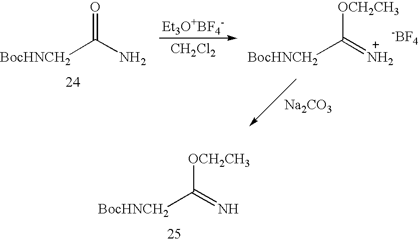
Triethyloxonium tetrafluoroborate is a powerful ethylating agent. It was reported that treatment of the amide with one equivalent of triethyloxonium tetrafluoroborate in methylene chloride at room temperature gave the imino ether. In addition, one equivalent of tetrafluoroboric acid (HBF₄) was generated during the reaction, which was considered a potential problem, because Boc protecting group is removed in strong acids. This reaction was performed in a large volume of solvent to dilute the acid generated in situ and the reaction was stopped after six hours even though there was trace of starting material remaining. Prolonging the reaction time was demonstrated to be detrimental to the product yield. Commercially available triethyloxonium tetrafluoroborate solution in methylene chloride (IM) in this reaction destroyed the starting material quickly and completely.

Boc-aminoacetioino ethyl ether 25 can not be purified by silica gel column chromatography, as it completely decomposed on the column. Thus, 25 was used without further purification.

After 25 was prepared, it was immediately reacted with L-serine methyl ester hydrochloride in methylene chloride at room temperature for 24 hours to afford methyl 2-(Boc-aminomethyl)-oxazoline-4-carboxylate (26). The ¹H-NMR spectrum showed the signals of methoxyl at δ 3.76 (s, 3 H, —OCH₃) and Boc at 1.43 (s, 9 H, t-butyl-O-). 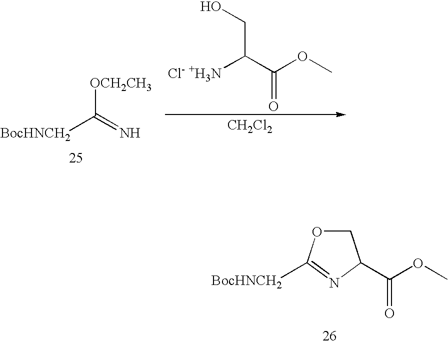
Oxazoline 26 is not stable in organic solvents or exposure to the air when dry. It is known in the art that when a pure oxazoline-containing amino acid methyl ester is exposed to the air for a couple of weeks, the oxazoline ring was open to form the corresponding dipeptide.

Dehydrogenation of 26 was achieved by treatment with four equivalents of CuBr₂/DBU/HTMA in methylene chloride at room temperature, and after 10 hours the reaction mixture was recharged with the reagent to react another day. After purification of the reaction mixture by partition and by silica gel column chromatography (hexane-EtOAc=4:1, 3:1 and 2:1), methyl 2-(Boc-aminomethyl)-oxazole-4-carboxylate (27) was obtained. The ¹H-NMR spectrum of 27 showed the aromatic proton peak of at δ 8.17 (s, 1 H), which confirmed the formation of an oxazole ring. The UV spectrum showed a maximum absorbance at 210 nm. 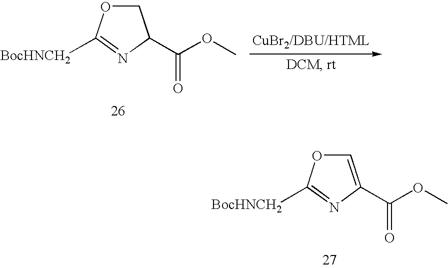
The dehydrogenation of a small quantity of 26 was satisfactorily achieved by the use of a CuBr₂/DBU/HMTA reagent.

2-Boc-aminomethyl-oxazole-4-carboxylic acid (28) was obtained from 27 by alkaline hydrolysis of 27 in THF/water solution for 2 hours in high yield (91%). The ¹H-NMR spectrum showed the signals of the oxazole aromatic proton at δ 8.39 (s, 1 H), and Boc at 1.44 (s, 9H, t-butyl-O—). The disappearance of methoxyl signal from the ¹H-NMR spectrum confirmed that the hydrolysis of 27 was complete.

The Boc protecting group of 28 was removed smoothly by TFA-DCM (1:1) in 45 min at room temperature.

After removal of Boc protecting group, without further purification, the residue was neutralized to re-protect the amino group by treatment with an excess of Fmoc-OSu and two equivalents of sodium carbonate in THF/water solution (2:1) to afford Fmoc-protected amino acid 2 (85%). The ¹H-NMR spectrum of the product showed the signals of Fmoc aromatic protons at δ 7.77-7.17 (m, 8H), confirmed the presence of Fmoc in 2. RP-HPLC analysis showed that the product has one peak. The ESI-MS measured molecular weight of 2 is consistent with the calculated mass.

Synthesis of 2-(2′-Fmoc-aminomethyl-oxazole-4′-yl)-thiazole-4-carboxylic Acid 3

The synthesis of 2-(2′-Fmoc-aminomethyl-oxazole-4′-yl)-thiazole-4-carboxylic acid 3 has been reported in Videnov et al., Angew. Chem. Int. Ed. Engl. 35 (13/14), 1503 (1996).

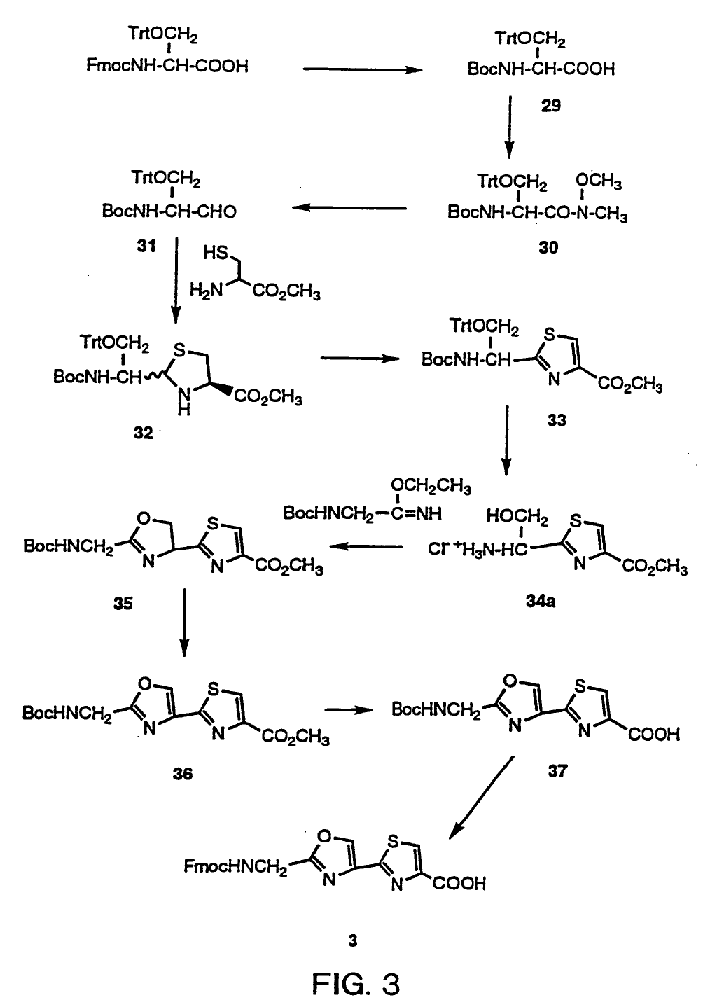

Referring to FIG. 3, a different strategy is disclosed herein: cyclocondensation of protected L-serinal (from protected L-serine) via its N-methoxyl-N-methyl amide with L-cysteine methyl ester afforded protected (Ser)-thiazolidine methyl ester, followed by dehydrogenation to give protected (Ser)-thiazole methyl ester. Then, protected (Ser)-thiazole methyl ester was deprotected and condensed with Boc-glycine imino ether to form the Boc-oxazolinyl thiazole which was dehydrogenated to afford the Boc-oxazolyl thiazole amino acid product.

The advantage of this strategy is that the first thiazole intermediate is easier to 20 synthesize, and is converted to the oxazolyl thiazole in two steps which minimized the loss of this intermediate.

Boc-Ser(Trt)-OH (29) was obtained by removing the Fmoc of commercially available Fmoc-Ser(Trt)-OH in diethylamine-methylene chloride (3:4) and then re-protecting the amino group of Ser(Trt)-OH with di-t-butyl dicarbonate in t-butanol aqueous solution (yield 78%). The ¹H-NMR spectrum of 29 showed the signals of Boc at δ 1.44 (s, 9 H, t-butyl-O—), trityl at 7.5-7.1 (m, 15 H) and serine at 10.86 (s, br, 1 H, —COOH), 5.35 (d, 1 H, J=8.3 Hz), 4.41 (m, 1 H), 3.61 (dd, 1 H, J=9.1 and 3.3 Hz), 3.38 (dd, 1 H, J=9.1 and 3.4 Hz), which confirmed the formation of 29.

The reason for converting Fmoc-Ser(Trt)-OH to Boc-Ser(Trt)-OH was to make the protecting groups compatible so that the protecting groups Boc and Trt could be removed simultaneously with TFA in later synthesis. Contrary to Boc, Fmoc could be removed by bases.

Boc-Ser(Trt)-OH (29) was then coupled with O,N-dimethylhydroxyl-amine hydrochloride under BOP activation in the presence of DIEA in methylene chloride for 20 min. to afford Boc-Ser(Trt)-N-methoxy-N-methyl amide (30). After purification by silica gel column chromatography (solvent system: hexane-EtOAc=5:1 and 3:1), 30 was obtained in high yield (98%). The ¹H-NMR spectrum of 30 showed the characteristic signals of the trityl group at δ 7.5-7.1 (m, 15 H), N-methoxy group at 3.57 (s, 3 H), N-methyl group at 3.18 (s, 3 H), and Boc at 1.43 (s, 9 H).

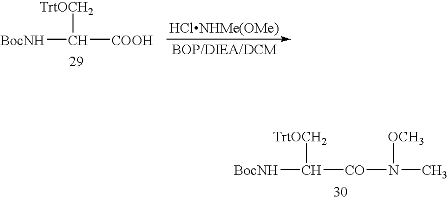

N^{α}-Boc-O-trityl-L-serinal (31) was prepared from 30 by the reduction of 30 with lithium aluminum hydride in anhydrous ethyl ether under argon at 0° C. for 30 min., followed by hydrolysis with aqueous potassium hydrogensulfate solution to produce 31 (yield 94%). The ¹H-NMR spectrum showed the aldehyde signal at δ 9.52 (s, 1 H, —CHO), which confirmed the formation of an aldehyde.

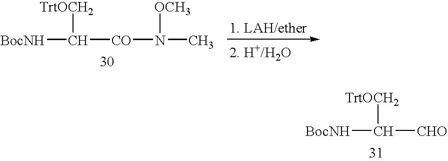

Without purification, 31 was used to condense with L-cysteine methyl ester in methylene chloride at room temperature, then in benzene to afford 32 (yield 95%). The ¹H-NMR spectrum showed the signals of trityl protons at δ7.5-7.1 (m, 15 H, trityl), methoxyl protons at 3.72 (s, 3 H, —OCH₃), and Boc at 1.45 (s, 9 H, t-butyl-O-).

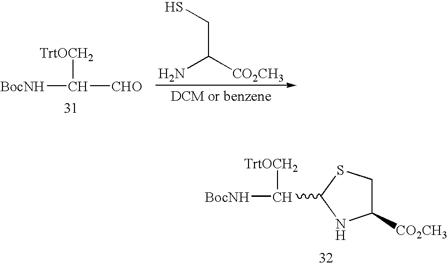

In case, 31 absorbed water from the air to form the undesired aldehyde hydrate, benzene was added to remove any water so that the equilibrium was shifted to force the cyclocondensation reaction to completion.

Thiazolidine 32 was dehydrogenated by active manganese dioxide in benzene at 50° C. for five hours to afford thiazole 33 (yield 59%). The ¹H-NMR spectrum and 2-D COSY experiment showed the signals of 33 at δ 8.06 (s, 1 H, aromatic), 7.5-7.1 (m, 15 H, trityl), 5.61 (d, 1 H, J=7.7 Hz), 5.17 (m, 1 H, α-H), 3.88 (s, 3 H, —O—CH₃), 3.78 (dd, 1 H, J=4.1 and 9.1 Hz, β-H), 3.46 (dd, 1 H, J=9.2 and 4.1 Hz, β-H′), and 1.42 (s, 9 H, t-butyl-O), confirming the formation of 33. 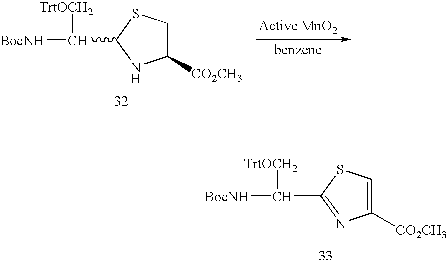
TLC monitoring of the dehydrogenation showed that one of the two thiazolidine diastereomers was quickly oxidized to the product, while the other was very resistant to oxidation. The resistant isomer needed a longer reaction time, and maybe additional oxidant for an efficient oxidation. Overall, longer reaction times should be avoided because the Boc protecting group is slightly thermo-labile.

After the protected (Ser)-thiazole methyl ester 33 was synthesized, both protecting groups (trityl and Boc) of 33 were removed in TFA/triethylsilane/DCM (50:10:40 v/v/v) for 45 min at room temperature. After purification, 34 was obtained (yield 90%). The ¹H-NMR spectrum showed the signals of an aromatic proton at δ 8.26 (s, 1H), α-H at 4.26 (dd, 1H, J=4.8 and 6.2 Hz,), methoxy group at 3.88 (s, 3H), β-H at 3.86 (dd, 1H, J=10.7 and 4.8 HZ), and β-H′ at 3.69 (dd, 1H, J=10.7 and 6.2 HZ), which confirmed the formation of 34. 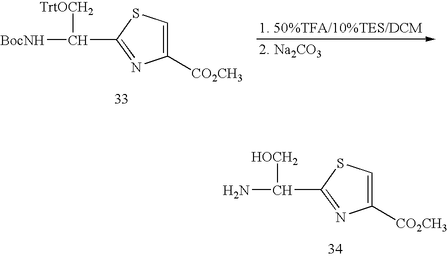
When deprotecting the Boc and trityl groups of 33, triethylsilane was added as a scavenger for the stable trityl cation so that the deprotection could proceed to completion. During purification, although triphenyl methane was washed away by methylene chloride, there was still a large quantity of water soluble impurity in the residue. It is simple and efficient to use a Sephadexg LH-20 column (eluent: methanol) to purify the residue.

To prepare the hydrochloride salt 34a of 34, 34 was dissolved in 1 M hydrochloric acid (aqueous solution), and concentrated to dryness in vacuo at room temperature.

The hydrochloride salt 34a was added to a solution of Boc-aminoacetimino ethyl ether 25 in methylene chloride and reacted for 24 hours at room temperature. After purification, 35 was obtained (yield 44%). The ¹H-NMR spectrun of 35 showed signals at δ 8.10 (s, 1H, aromatic), 5.53 (dd, 1H, J=7.9 and 9.6 Hz, oxazoline-H-4), 5.17 (s, br, 1H), 4.854.40 (m, 2H, oxazoline-H-5), 4.03 (d, 2H, J=5.7 Hz), 3.93 (s, 3H, —O—CH₃), and 1.46 (s, 9H, t-butyl-O—), which confirmed the formation of 35.

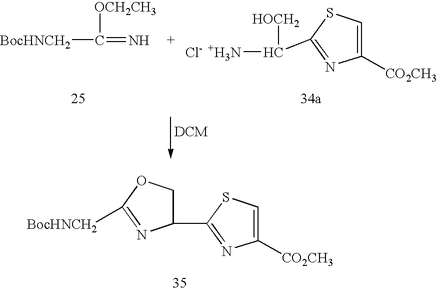

Methyl 2-(2′-Boc-aminomethyl-oxazole4′-yl)-thiazole-4-carboxylate (36) was obtained by dehydrogenation of 35 with nickel peroxide in benzene at 70° C. (yield 22%). The ¹H-NMR spectrum showed the signals of the thiazole and oxazole aromatic protons at δ 8.28 (s, 1H) and 8.15 (s, 1H), methoxy group at 3.95 (s, 3H), and Boc at 1.47 (s, 9H). 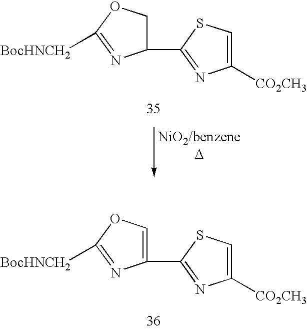
Dehydrogenation of oxazolines can be achieved by nickel peroxide in benzene by reflux for several hours to several days as reported in Evans et al. J. Org. Chem., 44 (4), 497 (1979) and Knight et al., Synlett, 1, 36 (1990), which are hereby incorporated by reference in their entireties into this disclosure.

The dehydrogenation of 36 by nickel peroxide proceeded smoothly. It was not necessary to reflux benzene solution for a long time. TLC monitoring (hexane-acetone=1:1) showed that the reaction finished within two hours at 70° C. The reaction had a low yield. For a fast and complete dehydrogenation, at least three equivalents of active oxygen from nickel peroxide is required. Prolonged reaction times should be avoided because the Boc protecting group is labile to heating, which will result in a low reaction yield. Also, active oxygen was lost by heating nickel peroxide for a long time. Therefore, if the reaction is not complete within two hours, a second or third charge of nickel peroxide is recommended.

Alkaline hydrolysis of 36 in THF/water (4:1) solution for two hours at room temperature afforded 37 (yield 90%). The ¹H-NMR spectrum showed the signals of the thiazole and oxazole protons at δ 8.46 (s, 1 H) and 8.34 (s, 1H), the methylene group at position 2 of the oxazole at 4.40 (s, 2H), and the Boc at 1.47 (s, 9H, t-butyl-O-).

The last step of the synthesis involved converting the amino protecting group of 37 from Boc to Fmoc. 37 was treated with 40% TFA in DCM for 60 min to completely remove the Boc protecting group. The residue from the deprotection of 37 was neutralized and was directly treated with Fmoc-OSu in a TIF/water (2:1) solution in the presence of sodium carbonate for two hours at room temperature to give Fmoc-protected amino acid 3 (yield 77%). The ¹H-NMR spectrum of 3 showed the signals of the thiazole and oxazole protons at δ 8.74 (s, 1H) and 8.38 (s, 1H), and the aromatic protons of Fmoc at 7.89-7.31 (m, 8H). RP-HPLC analysis showed that the product has one peak. The ESI-MS measured molecular weight of 3 is consistent with the calculated mass.

Compound 3 did not dissolve in DCM, ethyl ether, EtOAc, THF, methanol, ethanol, acetonitrile, HFIP, DMF, NMP or water. It could only dissolve in DMSO, or the solutions containing at least 50% DMSO in DMF or NMP.

Synthesis of Fmoc-alutamine (38) The N-α protection of L-glutanine with Fmoc was achieved by treatment with Fmoc-OSu in THF/water (2:1) solution in the presence of sodium carbonate overnight at room temperature to afford 38 (84%). The 1H-NMR spectrum of 38 showed the signals of the Fmoc aromatic protons at δ 7.89-7.20.

In this reaction, an excess of L-glutamine was used so that the purification of 38 was easier. After the reaction was finished, the reaction mixture was diluted with acidic aqueous solution. By filtration, the solid product 38 was collected and the excessive L-glutamine in solution was removed.

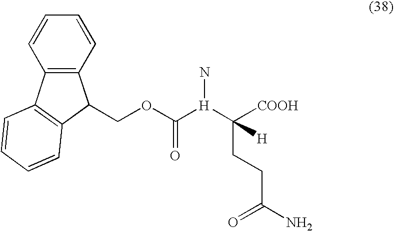

Experimental Section

General

Lithium aluminum hydride (LAH), N, O-dimethylhydroxylamine hydrochloride, tetrahydrofuran (anhydrous) (THF), potassium hydrogensulfate, L-cysteine methyl ester hydrochloride, diethylamine (DEA), and triethylsilane (TES) were purchased from Aldrich. N, N′-Dicyclohexylcarbodiimide (DCC), manganese (IV) oxide (activated), hexamethylenetetranine (HMTA), N, N-diisopropylethylamine (DIEA), nickel peroxide, 1,8-diazabicyclo [5.4.0] undec-7-ene (1,5-5) (DBU), triethyloxonium tetrafluoroborate, and cupric bromide were purchased from Fluka. Boc-glycine, glycine amide hydrochloride, benzotriazole-1-yl-oxy-tris(dimethylamino)-phosphonium hexafluorophosphate (BOP), trifluoroacetic acid (TFA), N-(9-Fluorenylmethyloxycarbonyl) oxysuccinimide (Fmoc-OSu), di-t-butyl dicarbonate, and L-serine methyl ester hydrochloride were from Advanced ChemTech. Dimethyl sulfoxide (DMSO), dichloromethane (DCM), and N, N-dimethylformamide (DMF) were from Burdick & Jackson. t-Butanol, L-glutamine, hexane, acetonitrile and diethyl ether (anhydrous) were from Fisher. Fmoc-Ser(Trt)-OH and benzotriazole-1-yl-oxy-tripyrrolidino-phosphonium hexafluorophosphate (PyBOP) were from Novabiochem.

Silica gel 60 for column chromatography (70-230 mesh) and silica gel TLC plates F254 (plastic or aluminum-backed sheet) were from E. Merck.

TLC developing solvent systems: (1) hexane-EtOAc; (2) hexane-acetone; (3) chloroform-MeOH-glacial HOAc (100:5:2 or 100:10:4). Methods for TLC visualization: 1. Examine the plate under UV light (254 nm); 2. Expose the plate to I₂ vapor in ajar for 5 min; 3. Spray the plate with a solution of 0.2% ninhydrin in 95% ethanol—10% aqueous acetic acid (95:5) and then heat at 110° C. for 5 min.

HPLC analysis was performed on a Waters HPLC system using a Vydac 218TP C18 10 □m reversed-phased column (250×4.6 mm) with a mobile-phase gradient: 40%-70% acetonitrile in 0.1% (v/v) TFA over 30 min, flow rate 1.0 ml/min, and UV detection at 215 and 290 nm. 18.2 MΩ water was produced by a Millipore Milli-Q plus system (Millipore, Bedford, Mass.).

UV spectra were recorded on a Hitachi U-2000 spectrophotometer.

ESI-MS were measured at Pfizer Central Research (Groton, Conn.) on a PE SCIEX API-100B LC/MS System (Foster City, Calif.). Mode: ESI, single quad, mn/z=300-2200, 4.2 sec/scan, flow rate: 200 μl/min, acetonitrile-water (50:50) in 0.1% TFA (v/v), and processed using BioMultiview 1.3 alpha program.

The ¹H NMR spectra were obtained on a modified EM-390 Varian spectrometer (EFT-90-30, Anasazi Instruments Inc., Indianapolis, Ind.), and processed with NUTS program (Win95 version, Acorn NMR Inc., Fremont, Calif.). Chemical shifts (δ) are given in ppm downfield from tetramethylsilane (TMS). Abbreviations for peak description are s=singlet, d=doublet, t=triplet, q=quartet, m=multiplet, and br=broad.

Synthesis of 2-(Fmoc-aminomethvl)-thiazole-4-carboxylic acid (1)

Boc-Gly-N-methoxy-N-methyl amide (19)

To a well stirred solution of Boc-Gly (21.02 g, 0.12 mole) and BOP (53.10 g, 0.12 mole) in 300 ml of DCM, was added DIEA (20.88 mi, 0.12 mole). After 10 min, a solution of O,N-dimethylhydroxylamine hydrochloride (14.05 g, 0.144 mole) and DIEA (31.32 ml, 0.18 mole) in 100 ml of DCM was added to above stirred solution. The reaction was monitored by TLC (silica gel, hexane-EtOAc =2:1). After 20 min, 200 ml of DCM was added to the reaction solution. The DCM solution was washed successively with 1 N aqueous hydrochloric acid solution (500 ml×4), saturated aqueous sodium bicarbonate solution (500 ml×3), and saturated aqueous sodium chloride solution (500 ml). The organic solution was dried with 5 g of magnesium sulfate overnight, filtered, and concentrated under reduced pressure. The residue was dissolved in a minimal volume of DCM, followed by addition of hexane until the solution became cloudy. The solution was warmed until it became clear and then kept to stay at room temperature to give colorless needles of 19 (21 g). Yield: 80%. TLC R_{f}=0.24 (hexane-EtOAc=2:1).¹H-NMR (90 MHz, CDCl₃) δ ppm: 5.21 (s, br, 1 H), 4.05 (d, 2 H, J=5.0 Hz), 3.70 (s, 3 H), 3.18 (s, 3 H), and 1.45 (s, 9 H).

Boc-glycinal (20)

Boc-Gly-N-methoxy-N-methylaride (19) (4.37 g, 20 mmole) in 150 ml of anhydrous THF was stirred in a ice-water bath under argon for 30 min. A solution of LAH in diethyl ether (1 M) (30 ml, 30 mmole) was added to the above well stirred solution by cannula under argon. The resulting solution was stirred for 30 min. A solution of potassium hydrogensulfate (4.77 g, 35 mmole) in 60 ml of water was gradually added to the reaction solution and stirred for 10 min. Organic solvents in the reaction mixture were evaporated under reduced pressure. An additional 60 ml of water was added to the aqueous residue, which was then extracted with DCM (100 ml×4). The combined DCM extracts were washed with 1 M hydrochloric acid solution (100 ml×4), saturated sodium bicarbonate solution (100 ml×2), and saturated sodium chloride solution (100 ml), dried with 4 g of magnesium sulfate overnight, and filtered. Evaporation of the solvent under reduced pressure left a yellowish oil 20 (2.83 g) which was used without further purification. Yield: 89%. TLC R_{f}=0.44 (hexane-EtOAc=1:1). ¹H-NMR (90 MHz, CDCl₃) δ ppm: 9.60 (s, 1 H), 5.26 (s, br, 1 H), 4.04 (d, 2 H, J=5.1 Hz), and 1.46 (s, 9 H).

Methyl 2-Boc-aminomethyl-thiazolidine-4-carboxylate (21)

To a stirred solution of Boc-glycinal (20) (2.83 g, 17.8 mmole) in 80 ml of DCM, was added (dropwise) a solution of L-Cys-OMe hydrochloride (3.86 g, 20 mmole) and DIEA (6.0 ml, 34 mmole) in 50 ml of DCM. The reaction finished within 5 min. Evaporation of the reaction mixture under reduced pressure afforded a residue. The residue was purified by silica gel column chromatography (50×cm, solvent system: hexane-EtOAc=2:1) to furnish 3.81 g of 21. Yield: 77%. TLC R_{f}=0.39 (hexane-EtOAc=1:1). ¹H-NMR (90 MHz, CDCl₃) δ ppm: 4.97 (s, br, 1 H), 4.9-4.6 (m, 1 H), 4.0-3.7 (m, 2 H), 3.76 (s, 3 H),3.4-2.7 (m, 3 H), 1.44 (s) and 1.43 (s) (9 H), which showed that 21 is a diastereomeric mixture (ca. 1:1).

Methyl 2-Boc-aminomethyl-thiazole-4-carboxylate (22)

A solution of 2-Boc-aminomethyl-thiazolidine-4-carboxylic methyl ester (21) (3.81 g, 13.8 mmole) in 150 ml of benzene was heated to 55° C. To above stirred solution, was added manganese (IV) oxide (activated) (30 g, 25 eq) and pyridine (1.5 ml). The reaction solution was stirred at 55° C. for 60 min. After initial filtration, the insoluble material was washed with DCM (100 ml×2). The combined filtrates were concentrated under reduced pressure and the residue was dissolved in minimal amount of DCM. Addition of hexane caused the DCM solution to become cloudy. The solution was warmed until it became clear and then kept at room temperature to give colorless needles of 22 (2.25 g). Yield: 60%. TLC R_{f}=0.12 (hexane-acetone=5:1). ¹H-NMR (90 MHz, CDCl₃) δ ppm: 8.10 (s, 1 H), 5.21 (s, br, 1 H), 4.63 (d, 2 H, J=6.4 Hz), 3.94 (s, 3 H), and 1.47 (s, 9 H). UV: λₘₐₓ (MeOH) 236 nm (ε 5880 M⁻¹ cm⁻¹).

2-Boc-aminomethyl-thiazole-4-carboxylic Acid (23)

To a stirred solution of methyl 2-Boc-aminomethyl-thiazolecarboxylate (22) (2.53 g, 9.2 mmole) in 80 ml of THF, was added 20 ml of 1 N sodium hydroxide aqueous solution. After 60 min, the spot corresponding to starting material disappeared on silica gel TlC (solvent system: hexane-acetone=1:1). The reaction solution was diluted with 200 ml of water and washed with DCM (300 ml×2 ). Acidification of the aqueous layer with 10% potassium hydrogensulfate to pH 3 was followed by extraction into EtOAc (200 ml×3). Concentration under reduced pressure of the dried EtOAc solution (magnesium sulfate) left a residue, which was recrystallized from MeOH-EtOAc-hexane to give a white powder 23 (2.21 g). Yield: 92%. TLC R_{f}=0.10 (CHCl₃-MeOH-HOAc=100:5:2). ¹H-NMR (90 MHz, DMSO-d₆) δ ppm: 8.28 (s, 1 H), 7.73 (s, br, 1 H), 4.37 (d, 2 H, J=5.8 H:z), and 1.41 (s, 9 H).

2-(Fmoc-aminomethyl)-thiazole-4-carboxylic Acid (1) 2-Boc-aminomethyl-thiazole-4-carboxylic acid (23) (2.21 g, 8.55 mmole) was dissolved in 120 ml of DCM-TFA (1:1) and stirred for 30 min. Removal of the solvent under reduced pressure was followed by neutralization of the residue with a solution of sodium carbonate (2 g, 19 mmole) in 40 ml of water, which was adjusted to pH 8 with additional solid sodium bicarbonate. Fmoc-OSu (4 g, 12 mmole) in 80 ml of THF was added to the resulting solution, and the mixture was stirred for 24 hours. The reaction mixture was concentrated under reduced pressure to remove THF and the residual liquid was washed with DCM (50 ml×4), and acidified to pH 3 with 1 N hydrochloric acid solution. The precipitate formed in the solution was collected by filtration, dried in vacuo, and recrystallized from DMF-0.1 N HCl (aqueous solution) to afford colorless needles (1, 3.13 g). Yield: 96%. TLC R_{f}=0.44 (CHCl3-MeOH-HOAc =50:5:2). RP-HPLC anlysis: retention time =10.45 min (average of two runs). ¹H-NMR (90 MHz, DMSO-d₆) δ ppm: 12.84 (s, br, 1H0, 8.30 (s, 1H), 7.89-7.17 (m, 8H), 5.15 (s, br, 1H), and 4.49-4.13 (m, 5H). ESI-MS (m/z): 381.3 [M+H]+, calculated monoisotopic mass 381.09.

Synthesis of 2-Fmoc-aminomethyl-oxazolecarboxylic Acid (2) Boc-glycine Amide (24)

To a stirred solution of glycine amide hydrochloride (11.06 g, 0.1 mole) and sodium hydroxide (4.0 g, 0.1 mole) in 25 ml of water and 50 ml of t-butanol, was added di-t-butyl dicarbonate (25 g, 0.11 mole) dropwise over a period of 15 min. After 15 min, an additional 50 nil of t-butanol was added to the reaction solution. The reaction was not stopped until the starting material disappeared, monitored by TLC (hexane-acetone=1:1) (ca. one hour). Then, the organic solvent was removed under reduced pressure and the residual solution was diluted with 50 ml of water. The aqueous solution was washed with petroleum ether (200 ml×3), acidified to pH 2 with 1 N hydrochloric acid solution, and extracted with EtOAc (200 ml×5). The dried EtOAc solution (magnesium sulfate) was filtered, and concentrated under reduced pressure to leave a residue, which was recrystallized from DCM-benzene to give colorless powders 24 (14.3 g). Yield: 82%. TLC R_{f}=0.36 (hexane-acetone=1:1). ¹H-NMR (90 MHz, CDCl₃) δ ppm: 6.00 (s, br), 5.62 (s, br), 5.16 (s, br), 3.80 (d, 2 H. J=5.8 Hz), and 1.46 (s, 9 H).

Boc-aminoacetimino Ethyl Ether (25)

To a stirred solution of Boc-glycine amide (24) (10.45 g, 60 mmole) in 600 ml of DCM under argon, was added triethyloxonium tetrafluoroborate (13.68 g, 95%, 68 mmole). The reaction solution was stirred under argon at room temperature for 6 hours and diluted with 400 ml of DCM, which was then poured into ice-cold sodium bicarbonate solution (1 M, 300 ml) and shaken well. The DCM layer was separated, dried over magnesium sulfate overnight, filtered, and concentrated under reduced pressure to afford an oily residue. This residue (crude 25) was used in next step without further purification. TLC R_{f}=0.23 (hexane-acetone =4:1). ¹H-NMR (90 MHz, CDCl₃) δ ppm: 5.03 (s, br, 1 H), 4.16 (q, 2 H, J =7.0 Hz), 3.72 (d, 2 H, J =6.3 Hz), 1.46 (s, 9H), and 1.29(t,3H,J=7.0Hz).

Methyl 2-(Boc-aminomethyl)-oxazoline-4-carboxylate (26)

To a stirred solution of all Boc-aminoacetimino ethyl ether (25) from the previous step in 200 ml of DCM, was added L-serine methyl ester hydrochloride (8.4 g, 54 mmole). After being stirred for 24 hours at room temperature, the reaction mixture was concentrated under reduced pressure to leave a residue. The crude product was used in next step without further purification. TLC R_{f}=0.46 (hexane-acetone =1:1). ¹H-NMR (90 MHz, CDCl₃) 8 ppm: 5.50 (s, br, 1 H), 4.94.4 (m, 3 H), 3.97 (d, 2 H, J =5.6 Hz), 3.76 (s, 3 H), and 1.43 (s, 9 H).

Methyl 2-Boc-aminomethvl-oxazole-4-carboxylate (27)

To a stirred suspension of cupric bromide (26.8 g, 0.12 mole) in 750 ml of DCM, were added hexamethylenetetramine (HMTA) (16.82 g, 0.12 mole) and 1,8-diazabicyclo[5.4.0]undec-7-ene (1,5-5) (DBU) (18 ml, 0.12 mole). After the resulting brown solution stirred for 10 min at room temperature, the crude methyl 2-Boc-aminomethyl-oxazoline-4-carboxylate (26) from previous step was added. After 10 hours, the reaction vessel was recharged with 80 mmole each of cupric bromide, HMTA, and DBU, and stirred for another day. The mixture was filtered and the filtrate was concentrated under reduced pressure to afford a residue, which was partitioned between 600 ml of EtOAc and 600 ml of saturated aqueous NH₄Cl-concentrated NH₄OH (1:1). The aqueous layer was then extracted with EtOAc (200 ml×3). The combined EtOAc extracts were washed with saturated aqueous NH₄Cl-concentrated NH₄OH (1:1) (150 ml×4), 1 M hydrochloric acid solution (300 ml×4), saturated sodium bicarbonate solution (300 ml), and saturated sodium chloride solution (300 ml), and dried by rnagnesium sulfate overnight. The dry EtOAc solution was filtered, and concentrated under reduced pressure to leave a residue, which was purified by silica gel column chromatography (10×4 cm, hexane-EtOAc=4:1, 3:1 and 2:1) to give 27 (1.34 g). TLC R_{f}=0.52 (hexane-acetone=1:1). ¹H-NMR (90 MHz, CDCl₃) δ ppm: 8.17 (s, 1 H), 5.50 (s, br, 1 H), 4.47 (d, 2 H, J=5.8 Hz), 3.89 (s, 3 H), and 1.44 (s, 3 H). UV: λₘₐₓ (MeOH) 210 nm (ε 7000 M⁻¹ cm⁻¹).

2-Boc-aminomethyl-oxazole4-carboxylic Acid (28)

A solution of methyl 2-Boc-aminomethyl-oxazole4-carboxylate (27) (1.42 g, 5.5 mmole) in 80 ml of THF and 20 ml of 1 M sodium hydroxide (aqueous solution) was stirred for 2 hours. The solution was concentrated under reduced pressure to remove THF. The residual solution was diluted with 100 ml of water, washed with DCM (50 ml×3), acidified to pH 2 by addition of 10% potassium hydrogensulfate (aqueous solution), and extracted with EtOAc (100 ml×5). The EtOAc solution was dried over magnesium sulfate overnight. The dried EtOAc solution was filtered, and concentrated under reduced pressure to give powders 28 (1.21 g). Yield: 91%. ¹H-NMR (90 MHz, CD₃0D) 8 ppm: 8.39 (s, 1 H), 4.36 (s, 2H), and 1.44 (s, 9H).

2-(Fmoc-aminomethyl)-oxazole-4-carboxylic Acid (2)

A solution of 2-Boc-aminomethyl-oxazole-4-carboxylic acid (28) in 50 ml of TFA-DCM (1:1) was stirred for 45 min and concentrated under reduced pressure to dry. Water 10 ml was added to the residue, which was neutralized to pH 7 by adding 1 M sodium hydroxide (aqueous solution), followed by addition of solid sodium carbonate (1.1 g, 10 mmole) and a solution of Fmoc-OSu (2.5 g, 7.4 mmole) in 100 ml of THF. After stirring for 17 hours, the reaction solution was concentrated under reduced pressure to remove THF, and diluted with 50 ml of water. The aqueous solution was washed with DCM (80 ml×3), acidified to pH 2 by adding concentrated hydrochloric acid, and extracted with EtOAc (150 ml×3). The combined EtOAc extracts were washed with 1 M hydrochloric acid solution (100 ml×4) and saturated sodium chloride aqueous solution (100 ml), and dried over magnesium sulfate overnight. The dry EtOAc solution was filtered, and concentrated under reduced pressure to a small volume (ca. 20 ml) and hexane (100 ml) was added to the residue. The white solid which formed was collected by filtration and then recrystallized with MeOH-water to give white powders (2, 1.55 g). Yield: 85%. TLC R_{f}=0.34 (CHCl₃-MeOH-HOAc =50:5:2). RP-HPLC analysis: retention time =10.93 min (average of two runs). ¹H-NMR (90 MHz, CD₃0D) δ ppm: 8.37 (s, 1H), 7.77-7.17 (m, 8H), and 4.42-4.09 (m, 5H). ESI-MS (m/z): 364.9 [M+H]⁺, calculated monoisotopic mass 365.11.

Synthesis of 2-(2′-Fmoc-aminomethyl-oxazole-4′-yl)-thiazole-4-carboxylic Acid (3)

Boc-Ser(Trt)-OH (29)

A solution of Fmoc-Ser(Trt)-OH (80 g, 0.14 mole) in 200 ml of DCM and 150 ml of diethylamine was stirred for three hours. The solution was concentrated under reduced pressure to leave a residue. The residue was dissolved in a solution of sodium hydroxide (5.6 g, 0.14 mole) in 50 ml of water and 200 ml of saturated aqueous sodium bicarbonate was added. The solution was washed with petroleum ether (300 ml×3) and diluted with 100 ml of t-butanol. To the resulting stirred solution, di-t-butyl dicarbonate (50 g, 0.22 mole) was added dropwise over a period of 30 min. After 15 min, an additional 100 ml of t-butanol was added to the reaction mixture, and it was stirred overnight. The solution was then diluted with 200 ml of water, washed with petroleum ether (400 ml×3), and cooled to 0° C. After three hours, the chilled solution was acidified to pH 3 with 1 N hydrochloric acid and extracted with EtOAc (600 ml×4). The combined EtOAc extracts was dried (magnesium sulfate) overnight, filtered, and concentrated under reduced pressure to leave a residue. The residue was purified by silica gel column chromatography (40×5 cm, solvent system: petroleum ether-EtOAc=4:1) to afford an oil, which was recrystallized from DCM-hexane to give crystals of (29) (49 g) . Yield: 78%. TLC R_{f}=0.38 (CHCl₃-MeOH-HOAc =100: 5:1). ¹H-NMR (90 MHz, CDCl₃) δ ppm: 10.86 (s, br, 1 H), 7.5-7.1 (m, 15 H), 5.35 (d, 1 H, J =8.3 Hz), 4.41 (m, 1 H), 3.61 (dd, 1 H, J=9.1 and 3.3 Hz), 3.38 (dd, 1 H, J=9.1 and 3.4 Hz), and 1.44 (s, 9 H).

Boc-Ser(Trt)-N-methoxy-N-methal Amide (30)

To a well stirred solution of Boc-Ser(Trt)-OH 29 (22.4 g, 50 mmole) and BOP (22.11 g, 50 mmole) in 100 ml of DCM, was added DIEA (8.7 ml, 50 mmole). The resulting solution was stirred at room temperature for 10 min. A solution of O, N-dimethylhydroxylamine hydrochloride (5.85 g, 60 mmole) and DIEA (15.66 ml, 90 mmole) in 60 ml of DCM was then added, and stirred for 20 min. The reaction mixture was concentrated under reduced pressure to afford a residue. The residue was purified by silica gel column chromatography (50×4 cm, solvent system: hexane-EtOAc =5:1 and 3:1) to give a colorless oil 30 (24 g). Yield: 98%. TLC R_{f}=0.30 (hexane-EtOAc =3:1). ¹H-NMR (90 MHz, CDCl₃) 8 ppm: 7.5-7.1 (m, 15 H), 5.48 (d, 1 H, J =8.8 Hz), 4.84 (m, 1 H), 3.57 (s, 3 H), 3.31 (d, 2 H, J =4.7 Hz), 3.18 (s, 3 H), and 1.43 (s, 9 H).

N^{α}-Boc-O-trityl-L-serinal (31)

Boc-Ser(Trt)-N-methoxy-N-methyl amide (30) (24 g, 49 mmole) in 400 ml of anhydrous diethyl ether was stirred in a ice-water bath under argon for 30 min. A commercially available LAH solution (1 M) in diethyl ether (100 ml, 0.1 mole) was added to the above well stirred solution by cannula under argon. The resulting solution was stirred for 30 min at 0° C. A solution of potassium hydrogensulfate (11.92 g, 87.5 mmole) in 200 ml of water was added to the reaction mixture, and it was stirred for 15 min. The reaction mixture was then diluted with 400 ml of diethyl ether. The organic layer was set aside and the aqueous layer was extracted with diethyl ether (300 ml×2). The combined ether extracts were washed with 1 M hydrochloric acid (500 ml×4), saturated sodium bicarbonate (500 ml×2), saturated sodium chloride (400 ml×2), dried with 16 g of magnesium sulfate overnight, and filtered. Concentration of the solvent under reduced pressure left an oil 31 (19.94 g) which was used in the next step without further purification. Yield: 94%. TLC R_{f}=0.58 (hexane-EtOAc=2:1). ¹H-NMR (90 MHz, CDCl₃) δ ppm: 9.52 (s, 1 H), 7.5-7.1 (m, 15 H), 5.27 (s, br, 1 H), 4.32 (m, 1 H), 3.55 (t,2 H), and 1.45 (s, 9 H).

Methyl 2-(1′-Boc-amino-2′-trityl-O-hydroxyethyl)-thiazolidine-4-carboxylate (32)

To a stirred solution of N-Boc-O-trityl-L-serinal (31) (19.94 g, 46.2 mmole) in 250 ml of DCM, was added a solution of L-Cys-OMe hydrochloride (9.4 g, 54 mmole) and DIEA (15 ml, 86 mmole) in 150 ml of DCM dropwise. TLC monitoring result showed that the reaction was not finished after stirring for 12 hours. Evaporation of the reaction solution under reduced pressure followed by addition of benzene (200 ml). The reaction was finished by the third cycle of addition and removal of benzene under reduced pressure to afford the desired product. The product was purified by silica gel column chromatography (40×5 cm, solvent system: hexane-EtOAc =4:1 and 3:1) to give 32 (24 g). 32 is a diastereomeric mixture which had two spots (ca. 1:1) with R_{f} 0.42 and 0.45 on TLC (hexane-EtOAc =2:1). Yield: 95%. ¹H-NMR (90 MHz, CDCl₃) δ ppm: 7.5-7.1 (m, 15 H), 5.2-4.7 (m, 2 H), 4.4-3.8 (m, 2 H), 3.72 (s, 3 H), 3.4-2.5 (m, 5 H), and 1.45 (s, 9 H).

Methyl 2-(1′-Boc-amino-2′-trityl-O-hydroxyethyl)-thiazole-4-carboxylate (33)

30 A solution of methyl 2-(1′-Boc-amino-2′-trityl-O-hydroxyethyl)-thiazolidine-4-carboxylate (32) (24 g, 43.7 mmole) in 400 ml of benzene was heated to 50° C. To above stirred solution, was added manganese (IV) oxide (activated) (118 g, 30 eq) and pyridine (4 ml). The reaction solution was stirred at 50°-55° C. for 5 hours (one of the isomers was quickly oxidized to the product, while the other was very resistant to the oxidation conditions). After filtration, the insoluble material was washed with DCM (100 ml×2). The combined filtrates were concentrated under reduced pressure and the residue was purified by silica gel column chromatography (40×5 cm, hexane-EtOAc =4:1) to give 33 (14 g). Yield: 59%. TLC R_{f}=0.48 (hexane-EtOAc =2:1). ¹H-NMR (90 MHz, CDCl₃) δ ppm: 8.06 (s, 1 H), 7.5-7.1 (m, 15 H), 5.61 (d, I H, J =7.7 Hz), 5.17 (m, 1 H), 3.88 (s, 3 H), 3.78 (dd, 1 H, J=4.1 and 9.1 Hz), 3.46 (dd, 1 H, J=9.2 and 4.1 Hz), and 1.42 (s, 9 H).

Methyl 2-(1′-amino-2′-hydroxyethyl)-thiazole-4-carboxylate (34)

Methyl 2-(1′-Boc-amino-2′-trityl-O-hydroxyethyl)-thiazole-4-carboxylate 33 (14 g, 25 mmole) was added to 200 ml of TFA/triethylsilane/DCM (50:10:40) and stirred for 45 min. The solution was concentrated under reduced pressure to leave a residue, to which 150 ml of 0.1 M hydrochloric acid was added. This acidic solution was washed with DCM (100 ml×4), neutralized to pH 9 by adding saturated sodium carbonate (aqueous solution), and concentrated to dry at room temperature in vacuo. The residue was purified on a Sephadex LH-20 column (50×2.5 cm) (eluent: methanol) to afford 34 (4.5 g). Yield: 90%. TLC R_{f}=0.16 (CHCl₃-MeOH-HOAc =50: 5: 2). ¹H-NMR (90 MHz, CD₃0D) 6 ppm: 8.26 (s, 1H), 4.26 (dd, 1H, J =4.8 and 6.2 Hz), 3.88 (s, 3H), 3.86 (dd, 1H, J =10.7 and 4.8 Hz), and 3.69 (dd, 1H, J =10.7 and 6.2 HZ).

Methyl 2-(2′-Boc-aminomethyl-oxazoline-4′-yl)-thiazole-4-carboxylate (35)

Methyl 2-(1′-amino-2′-hydroxyethyl)-thiazole-4-carboxylate (34) (4.5 g, 22.5 mmole) was dissolved in 30 ml of 1 M hydrochloric acid (aqueous solution) and concentrated at room temperature to dry in vacuo. The hydrochloride salt 34a was then added to a solution of Boc-aminoacetimino ethyl ether (see preparation of 25, from Boc-glycine amide 10.45 g, 60 mmole) in 100 ml of DCM. The reaction mixture was stirred at room temperature for 24 hours, followed by removal of the solvent under reduced pressure. The residue was purified by silica gel column chromatography (40×5 cm, solvent system: hexane-acetone gradient) to yield 35 (3.43 g). Yield: 44%. TLC R_{f}=0.39 (hexane-acetone=1:1). ¹H-NMR (90 MHz, CDCl₃) δ ppm: 8.10 (s, 1H), 5.53 (dd, 1H, J=7.9 and 9.6 Hz), 5.17 (s, br, 1H), 4.85-4.40 (m, 2H), 4.03 (d, 2H, J=5.7 Hz), 3.93 (s, 3H), and 1.46 (s, 9H).

Methyl 2-(2′-Boc-aminomethyl-oxazole-4′-yl)-thiazole-4-carboxylate (36)

To a stirred solution of methyl 2-(2′-Boc-aminomethyl-oxazoline-4′-yl)-thiazole-4-carboxylate (35) (3.43 g, 10 mmole) in 100 ml of benzene at 70° C., nickel peroxide (5 g, 1.6 eq. of active O₂) was added and stirred for 10 hours at 70° C. The solid material was removed by filtration and the filtrate was concentrated under reduced pressure to leave a residue, which was purified by silica gel chromatography (10×2.5 cm, solvent system: hexane-acetone =2:1). The fractions containing 36 were pooled and recrystallized in DCM-MeOH to yield 36 (0.76 g). Yield: 22%. TLC R_{f}=0.70 (hexane-acetone=1:1). ¹H-NMR (90 MHz, CDCl₃) δ ppm: 8.28 (s, 1H), 8.15 (s, 1H), 5.07 (s, br, 1H), 4.48 (d, 2H, J =6.2 Hz), 3.95 (s, 3H), and 1.47 (s, 9H).

2-(2′-Boc-aminomethyl-oxazole-4′-yl)-thiazole-4-carboxylic Acid (37)

Methyl 2-(2′-Boc-aminomethyl-oxazole-4′-yl)-thiazole-4-carboxylate (36) (0.76 g, 2.2 mmole) was dissolved in 30 ml of THF and 20 ml of 1 M sodium hydroxide (aqueous solution) and stirred at room temperature for 2 hours. The solution was concentrated under reduced pressure to remove the THF. The residue was diluted with 100 ml of water, washed with DCM (50 ml×3), acidified to pH 2 by adding 10% potassium hydrogensulfate (aqueous solution), and extracted with EtOAc (100 ml×4). The EtOAc extracts were dried (magnesium sulfate) overnight, filtered, and concentrated under reduced pressure to a small volume to give a powder 37 (0.65 g). Yield: 90%. ¹H-NMR (90 MHz, CD₃OD) 6 ppm: 8.46 (s, 1 H), 8.34 (s, 1H), 4.40 (s, 2H), and 1.47 (s, 9H).

2-(2′-Fmoc-aminomethyl-oxazole-4′-yl)-thiazole-4-carboxylic Acid (3)

2-(2′-Boc-aminomethyl-oxazole-4′-yl)-thiazole-4-carboxylic acid (37) (0.65 g, 2 mmol) was added in 30 ml of 40% TFA in DCM and stirred for 60 min. The solvent was removed by concentration under reduced pressure. The residue was diluted with 10 ml of water, which was neutralized to pH 7 by adding 1 M sodium hydroxide (aqueous solution), followed by addition of solid sodium carbonate (0.42 g, 4 mmole) and a solution of Fmoc-OSu (1 g, 3 mmole) in 60 ml of THF. After stirring for 2 hours (white solid precipitated), the reaction mixture was concentrated under reduced pressure to remove the THF, and diluted with 30 ml of water. The aqueous solution was washed with DCM (50 ml×3), and acidified to pH 3 by adding 10% potassium hydrogensulfate (aqueous solution). The white solid which formed was collected by filtration. This product was insoluble in DCM, ether, EtOAc, THF, methanol, ethanol, acetonitrile, HFIP, DMF, NMP and water. It was soluble in DMSO or the solutions containing more than 50% DMSO in DMF or NMP. The product was recrystallized in DMSO-water to give a fine white powder (0.69 g). Yield: 77%. RP-HPLC analysis: retention tLneime=14.18 min (average of two runs). ¹H-NMR (90 MHz, DMSO-d6) δ ppm: 8.74 (s, 1H), 8.38 (s, 1H), 7.89-7.31 (m, 8H), and 4.394.30 (m, 5H). ESI-MS (m/z): 448.2 [M +H]+, (calculated monoisotopic mass 448.10), 470.2 [M+Na]⁺, 486.2 [M+K]⁺. Compound (3) exhibits unexpected properties.

Synthesis of Fmoc-glutamine (38)

To a stirred solution of L-glutamine (1.75 g, 12 mmole) and sodium carbonate (1.27 g, 12 mmole) in 30 ml of water, was added a solution of Fmoc-OSu (3.0 g, 8.9 mmole) in 60 ml of THF. After stirring overnight the mixture was concentrated under reduced pressure to remove the THF. The residue was diluted with 100 ml of 1 N hydrochloric acid . The white solid was collected by filtration and recrystallized in DMF-0.1 N hydrochloric acid aqueous solution to afford a white powder (38, 2.76 g). Yield: 84%. ¹H-NMR (90 MHz, DMSO-d6) □ ppm: 12.47 (1H, s), 7.89-7.20 (10 H, m), 6.68 (1H, s, br), 4.22-3.90 (4H, m), and 2.25-1.86 (4H, m).

Combinatorial Synthesis

In one embodiment, the present invention relates to the generation of a synthetic combinatorial library of at least two compounds, each compound within the library being derived from the solid phase combinatorial synthesis of at least one compound selected from the group consisting of:

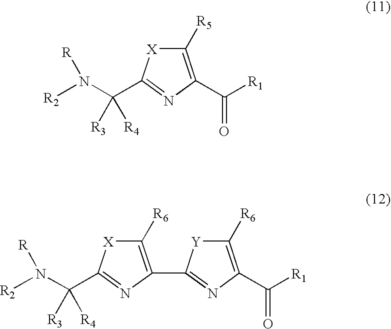

where R=H, a naturally occurring or synthetic L or D amino acid, Tert-butyloxycarbonyl (Boc), 9-fluorenylmethoxycarbonyl (Fmoc), carbobenzoxy (Z), Benzozyl (Bz), and other like amino protecting groups;

where R₁═OH, alkyl esters, aromatic esters such as methyl, ethyl, t-butyl and benzyl, activated esters such as pentafluorophenyl, nitrophenyl, N-hydroxysuccinimide, acid chlorides, fluorides, organic salts, such as cyclohexyamines (CHA), amides, an amide bonded to a linker such as a diamine, or an insoluble support for use in solid phase synthesis;

where R₂═H, a C₁-C₁₀ alky, or an aromatic ring;

where R₃₋₄═H, or a C₁-C₁₀ alkyl;

where R₅₋₆═H, C₁-C₁₀ alkyl, a heterocylic ring, an aliphatic or aromatic ring, a functional group such as an amine, an alchohol, a halide or an organometallic complex;

where X═oxygen (O) or sulfur (S);

where Y═oxygen (O) or sulfur (S);

wherein at least one of the compounds selected from the group of 11 and 12 forms an amide bond with at least one of the compounds selected from the group of 11 and 12 or a naturally occurring or synthetic amino acid.

In another embodiment of the invention, at least one of the compounds 11 and 12 is combined with a natural amino acid in a combinatorial synthesis to yield a naturally occurring antibiotic compound.

In a preferred embodiment of the invention, at least one the compounds selected from the group 11 and 12 is combined with a natural amino acid in combinatorial synthesis to yield a microcin B17 fragment.

The following compounds are compounds especially believed to be suitable for purposes of the invention.

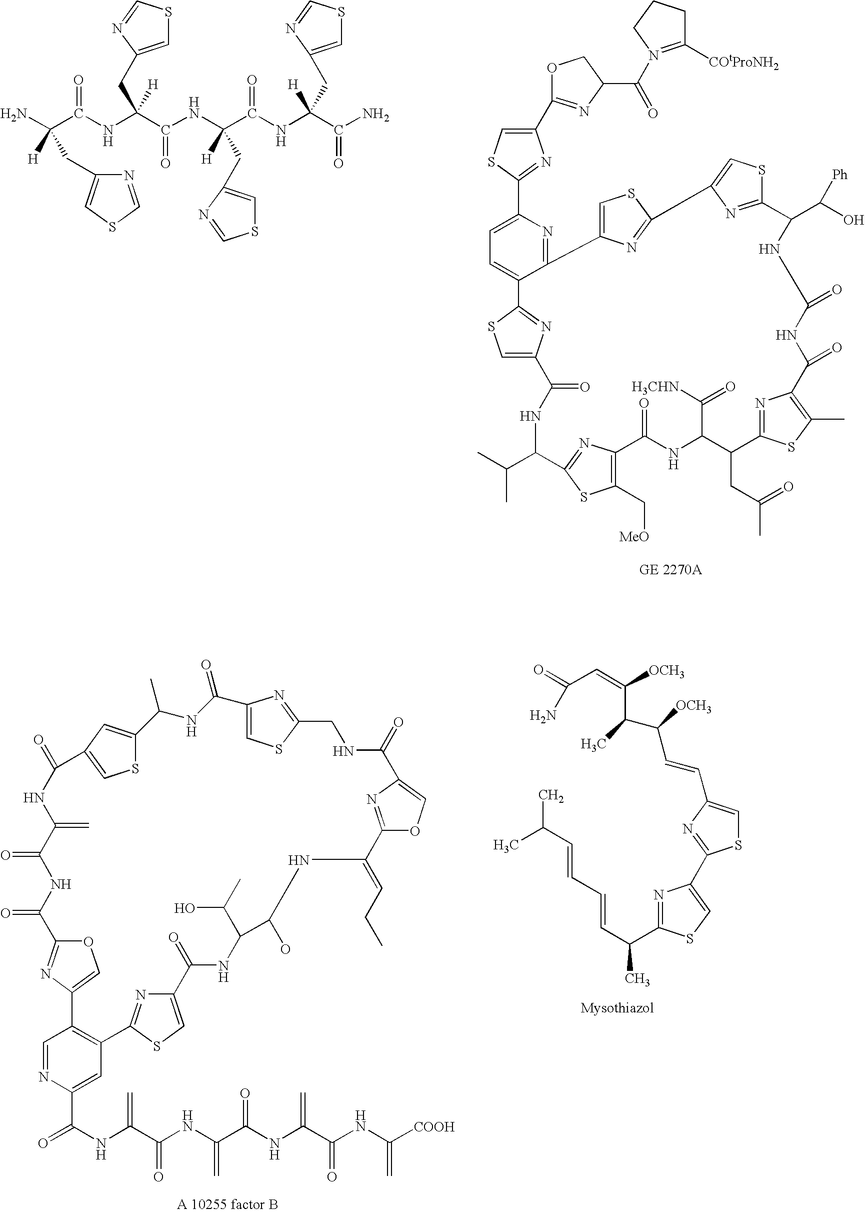

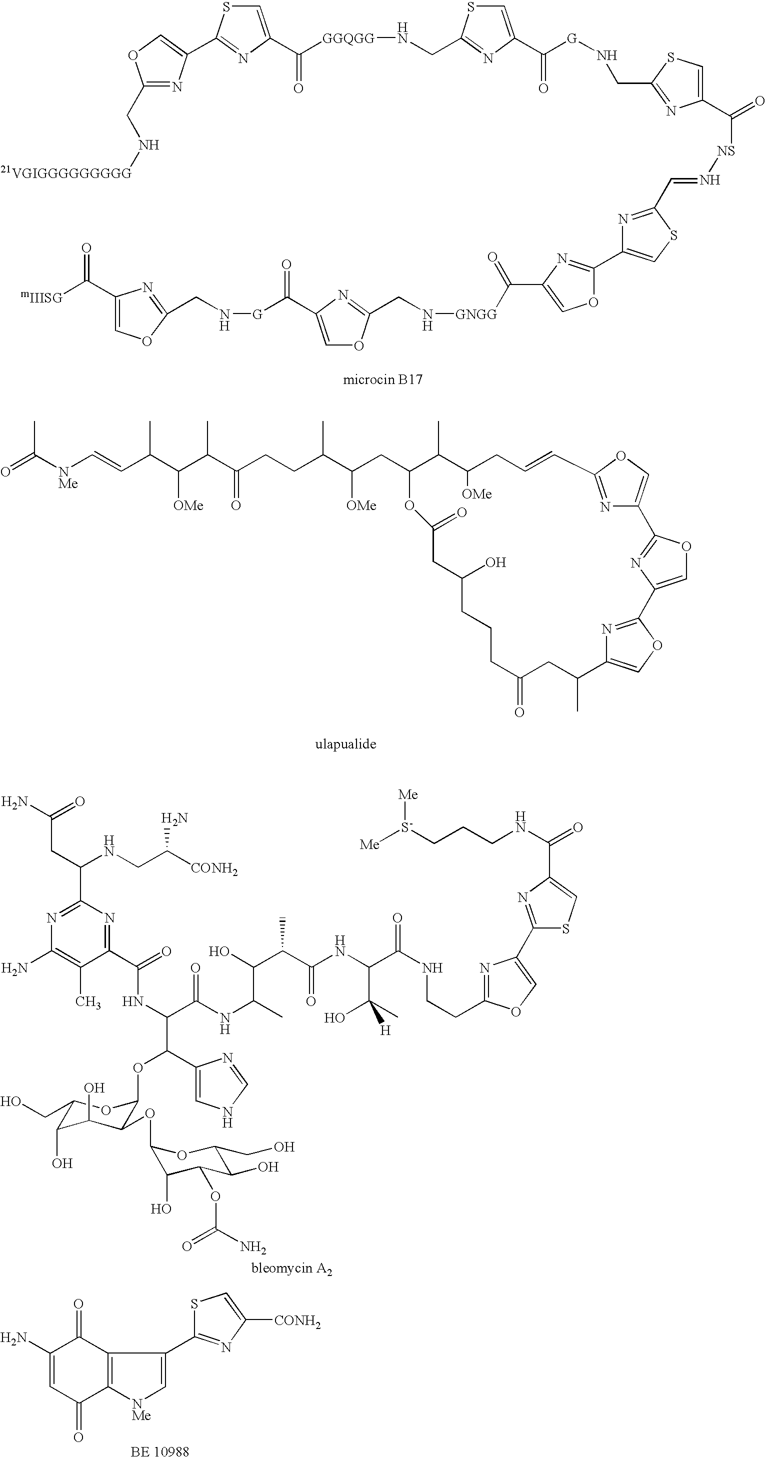

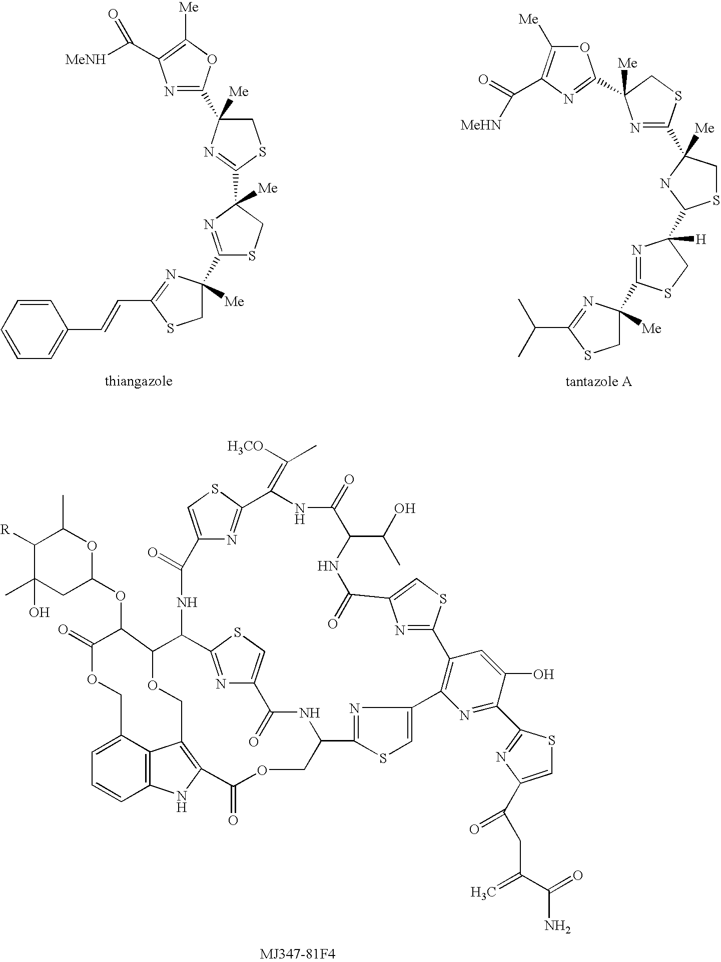

Another embodiment of the invention relates to the generation of a synthetic combinatorial library of at least two compounds, each compound within the library being derived from the solid phase combinatorial synthesis of at least one compound selected from the group consisting of:

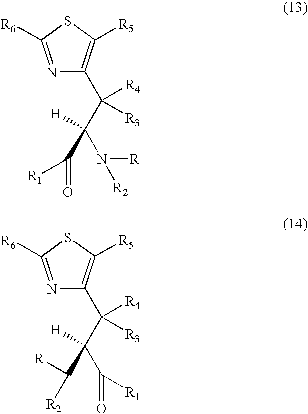

where R═H, a naturally occurring or synthetic L or D amino acid, Tert-butyloxycarbonyl (Boc), 9-fluorenylmethoxycarbonyl (Fmoc), carbobenzoxy (Z), Berizozyl (Bz), and other like amino protecting groups;

where R₁═OH, alkyl esters, aromatic esters such as methyl, ethyl, t-butyl and benzyl, activated esters such as pentafluorophenyl, nitrophenyl, N-hydroxysuccinimide, acid chlorides, fluorides, organic salts, such as cyclohexyamines (CHA), amides, an amide bonded to a linker such as a diamine, or an insoluble support for use in solid phase synthesis;

where R₂═H, a C₁-C₁₀ alkyl or an aromatic ring;

where R₃₋₄═H, or a C₁-C₁₀ alkyl;

where R₅₋₆═H, C₁-C₁₀ alkyl, a heterocylic ring, an aliphatic or aromatic ring, a functional group such as an amine, an alchohol, a halide or an organometallic complex;

wherein at least one of the compounds selected from the group of 13 and 14 forms an amide bond with at least one of the compounds selected from the group of 13 and 14 or a naturally occurring or synthetic amino acid.

Because libraries can be screened while still bound to the resin, additional embodiments of the invention include any of the above-described libraries bound to a solid-phase resin.

Although certain structures have been shown, enantiomers of those structures are within the scope of the invention.

In yet another embodiment of the invention, a method for the preparation of a library of antibiotic compounds comprises coupling an amino protected first amino acid to a resin, the first amino acid selected from the group consisting of:

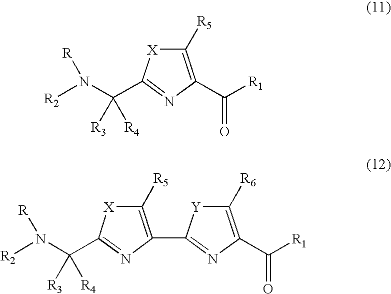

where R═H, a naturally occurring or synthetic L or D amino acid, Tert-butyloxycarbonyl (Boc), 9-fluorenylrnethoxycarbonyl (Fmoc), carbobenzoxy (Z), Benzozyl (Bz), and other like amino protecting groups;

where R₁═OH, alkyl esters, aromatic esters such as methyl, ethyl, t-butyl and benzyl, activated esters such as pentafluorophenyl, nitrophenyl, N-hydroxysuccinimide, acid chlorides, fluorides, organic salts, such as cyclohexyamines (CHA), amides, an amide bonded to a linker such as a diamine, or an insoluble support for use in solid phase synthesis;

where R₂═H, a C₁-C₁₀ alkl or an aromatic ring;

where R₃₋₄═H, or a C₁-C₁₀ alkyl;

where R₅₋₆═H, C₁-C₁₀ alkyl, a heterocylic ring, an aliphatic or aromatic ring, a functional group such as an amine, an alchohol, a halide or an organometallic complex;

where X═oxygen (O) or sulfur (S);

where Y═oxygen (O) or sulfur (S);

removing the protecting group of the first amino acid, coupling an amino protected second amino acid selected from the group consisting of 11 and 12 or a naturally occurring or synthetic amino acid and cyclizing the compounds selected from the group consisting of 11 and 12 or a naturally occurring or synthetic amino acid from the step of coupling.

In yet another embodiment of the invention, a method for the preparation of a library of antibiotic compounds comprises coupling an amino protected first amino acid to a resin, the first amino acid selected from the group consisting of:

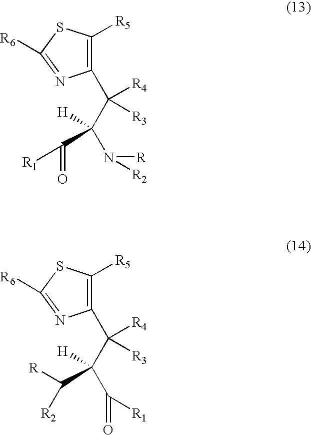

where R═H, a naturally occurring or synthetic L or D amino acid, Tert-butyloxycarbonyl (Boc), 9-fluorenylmethoxycarbonyl (Fmoc), carbobenzoxy (Z), Benzozyl (Bz), and other like amino protecting groups;

where R₁═OH, alkyl esters, aromatic esters such as methyl, ethyl, t-butyl and benzyl, activated esters such as pentafluorophenyl, nitrophenyl, N-hydroxysuccinimide, acid chlorides, fluorides, organic salts, such as cyclohexyamines (CHA), amides, an amide bonded to a linker such as a diamine, or an insoluble support for use in solid phase synthesis;

where R₂═H, a C₁-C₁₀ alkyl or an aromatic ring;

where R₃₋₄═H, or a C₁-C₁₀ alkyl;

where R₅₋₆═H, C₁-C₁₀ alkyl, a heterocylic ring, an aliphatic or aromatic ring, a functional group such as an amine, an alchohol, a halide or an organometallic complex; removing the protecting group of the first amino acid, coupling an amino protected second amino acid selected from the group consisting of 3 and 4 or a naturally occurring or synthetic amino acid, and cyclizing the compounds selected from the group consisting of 3 and 4 or a naturally occurring or synthetic amino acid from the step of coupling.

Results and Discussion

Synthesis of Library One (L1)

Table 1 shows the pair of enantiomeric thiazole-containing unnatural amino acids, 13 and 14, that were chosen as the building blocks to synthesize a library of sixteen tetrapeptide amides. The thiazole ring is on the side chain of the amino acids.

TABLE 1
The Sequences of peptide amides of L1

| | | | | | | | |
|---|---|---|---|---|---|---|---|
| 1 | DDDD- | 5 | DDLL- | 9 | DLLL- | 13 | DLDD- |
| | NH₂^{a} | | NH₂ | | NH₂ | | NH₂ |
| 2 | DDDL- | 6 | DDLD- | 10 | DLDL- | 14 | DLLD- |
| | NH₂ | | NH₂ | | NH₂ | | NH₂ |
| 3 | LDDL- | 7 | LDLL- | 11 | LLDL- | 15 | LLLD- |
| | NH₂ | | NH₂ | | NH₂ | | NH₂ |
| 4 | LDDD- | 8 | LDLD- | 12 | LLLL- | 16 | LLDD- |
| | NH₂ | | NH₂ | | NH₂ | | NH₂ |

D: D-(3)-(4-thiazolyl) alanine (R)
L: L-(3)-(4-thiazolyl) alanine (S)
^{a}The linkage between monomers is amide bond.

| | |
|---|---|
| | (13) |
| 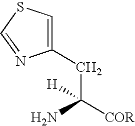 | |
| L-(3)-(4-thiazolyl)alanine | |
| | (14) |
| 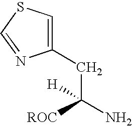 | |

D-(3)-(4-thiazolyl)alanine

Peptide amide was synthesized on MBHA resin by Boc strategy. Sixteen syringes of a MULTIBLOCK simultaneous multiple peptide synthesizer were used for this library synthesis.

Boc-amino acids were coupled to the resin by four equivalents of amino acid under the activation of four equivalents of DCC in DCM for 60 min. Ninhydrin test showed that the coupling reaction was satisfactory and there was no need for a second coupling. Boc was removed in 40% TFA in DCM for 30 min.

After the sequences were synthesized, the resin was washed and dried in vacuo. The dried peptide resin was cleaved with HF at 0° C. for 60 min without adding any scavenger.

HPLC analysis results showed that each individual compound had one peak at 215 nm, indicating that each compound in the library is in high purity.

Because the process and conditions of library synthesis was identical to individual compound in this library and the compounds are either enantiomers or diastereomers, compound L1-4 (LDDD-NH₂) was chosen as an example to analyze its structure. ¹H-NMR spectrum of L1-4 showed the signals of four thiazolyl aromatic protons at δ 8.88-8.87 (4H, m), 7.29 (1H, d, J=1.85 Hz), 7.26 (1H, d, J=1.82 Hz), 7.22 (1H, d, J=1.83 Hz), and 7.16 (1H, d, J=1.82 Hz), indicating that the thiazole ring was intact under solid phase synthesis conditions and HF cleavage. The ESI-MS showed the molecular ion peak at 634.1 [M+1]⁺, (calculated monoisotopic mass 634.11), confirmed the integrity of this peptide. The UV spectrun showed a typical maximum absorbance of thiazole ring at 238 nm (31).

Synthesis of Library Two (L2)

2-Fmoc-aminomethyl-thiazole-4-carboxylic acid (1), 2-Fmoc-amino-methyl-oxazole-4-carboxylic acid (2), and 2-(2′-Fmoc-aminomethyloxazole-4′-yl)-thiazole-4-carboxylic acid (3) were synthesized as previously discussed.

TABLE 2
The sequences of the compounds in library two (L2)

| | | | |
|---|---|---|---|
| 1 | Ac-AGA-NH—(CH₂)₃—NH₂^{a} | 9 | Ac-AGA-NH—(CH₂)₃—NH₂ |
| 2 | Ac-AGA-NH—(CH₂)₃—NH₂ | 10 | Ac-AGA-NH—(CH₂)₃—NH₂ |
| 3 | Ac-AGA-NH—(CH₂)₃—NH₂ | 11 | Ac-AGA-NH—(CH₂)₃—NH₂ |
| 4 | Ac-AGA-NH—(CH₂)₃—NH₂ | 12 | Ac-AGA-NH—(CH₂)₃—NH₂ |
| 5 | Ac-AGA-NH—(CH₂)₃—NH₂ | 13 | Ac-AGA-NH—(CH₂)₃—NH₂ |
| 6 | Ac-AGA-NH—(CH₂)₃—NH₂ | 14 | Ac-AGA-NH—(CH₂)₃—NH₂ |
| 7 | Ac-AGA-NH—(CH₂)₃—NH₂ | 15 | Ac-AGA-NH—(CH₂)₃—NH₂ |
| 8 | Ac-AGA-NH—(CH₂)₃—NH₂ | 16 | Ac-AGA-NH—(CH₂)₃—NH₂ |

^{a}The linkage between monomers is amide bond.
A: 2-Aminomethyl-thiazole-4-carboxylic acid
B: 2-Aminomethyl-oxazole-4-carboxylic acid
C: 2-(2′-Aminomethyl-oxazole-4′-yl)-thiazole-4-carboxylic acid
G: glycine

| | |
|---|---|
| A: | 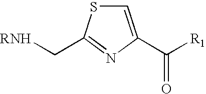 |
| B: | 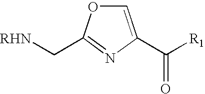 |
| C: | 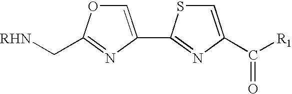 |

where R═H and R₁═OH

The following compounds from the library L2 were identified by using UV spectrum, ESI-MS and RP-HPCL technology:

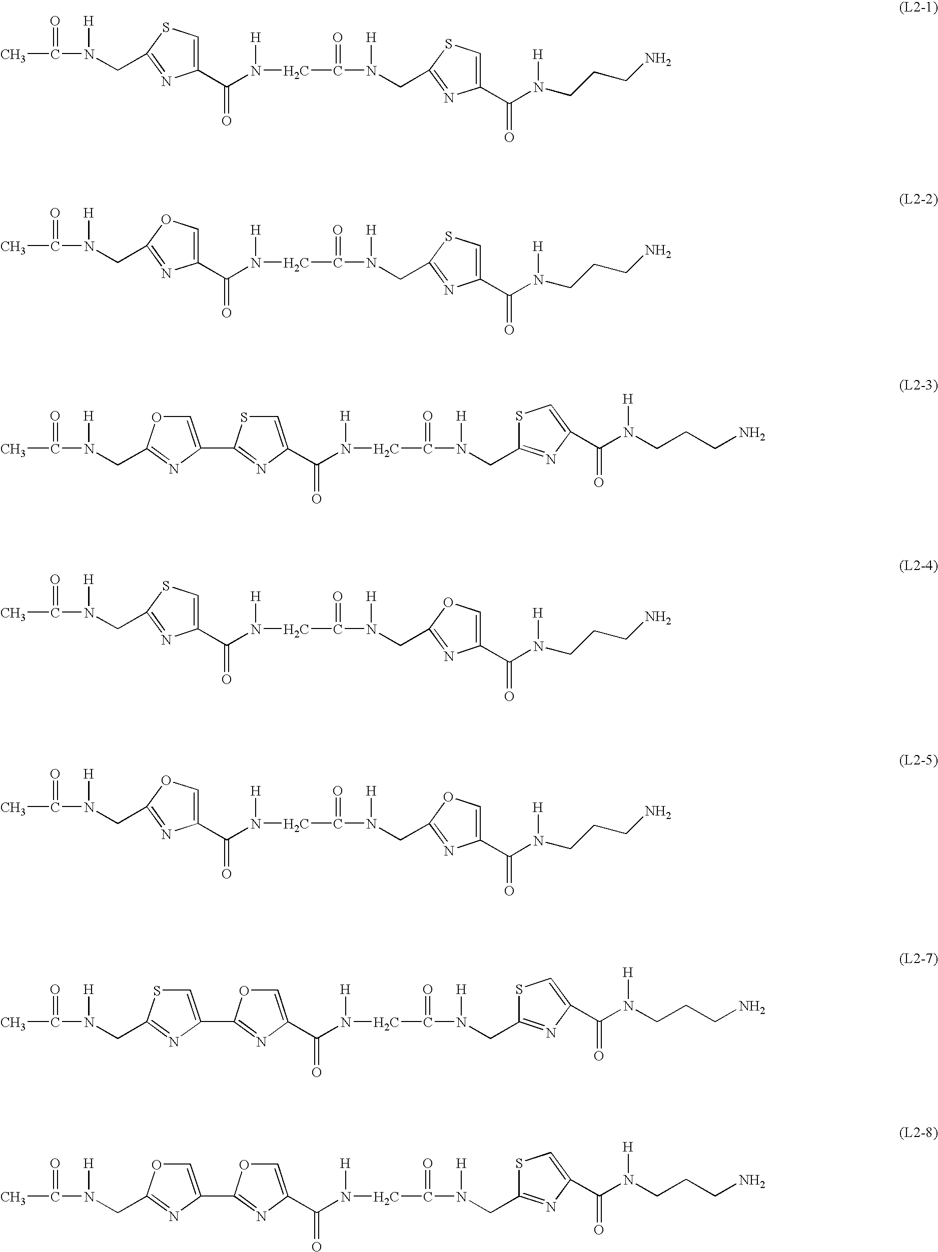

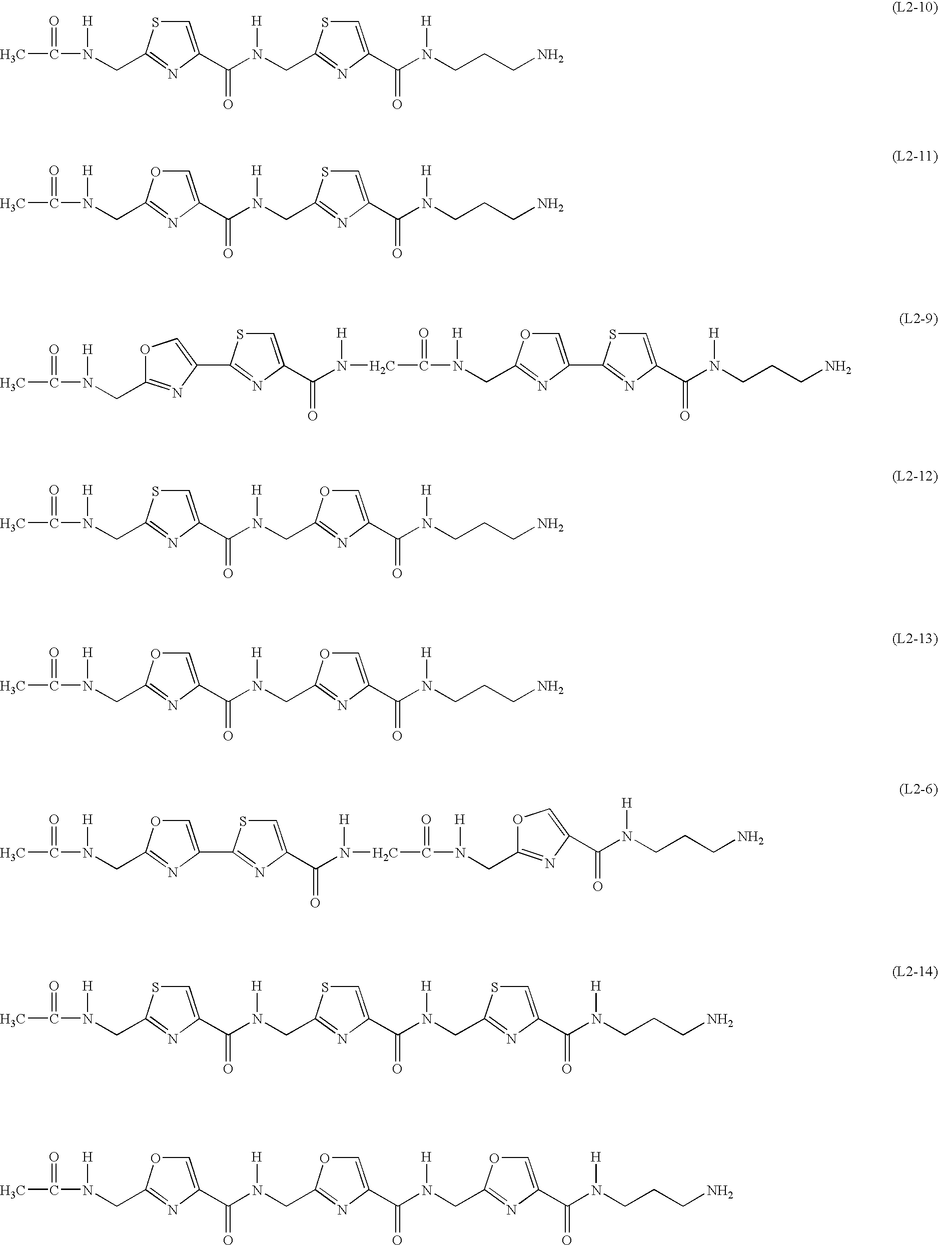

Compounds L2-1 to L2-9 were the combinations of three building blocks with a glycine insertion between two building blocks in each compound. The glycine addition was designed to increase the flexibility of the peptide. L2-10 to L2-13 were the combinations of building blocks 1 and 2, in which 3 was not used. Otherwise, it was thought that the structures of peptides would be too rigid. Compounds L2-14 and L2-15 were designed to compare with L2-10 and L2-13.

This library was synthesized on 1,3-diaminopropane trityl resin by Fmoc strategy. Therefore, the C-terminal of each compound has a propylamine unit. Fifteen syringes of a MULTIBLOCK simultaneous multiple peptide synthesizer were used for this library synthesis.

Coupling reaction was performed by 1,5 equivalents of Fmoc-amino acid: BOP: HOBt: DIEA (1:1:1:1) with 10-min preactivation before coupling. Although it was shown that the coupling completion was very rapid (less than 10 min) (16), a long reaction time (60 min) was applied to coupling for only 1.5 equivalents of amino acids was added in the reaction. Ninhydrin test indicated that the coupling was satisfactory. Fmoc was removed in 20% piperidine in NMP for 20 min.

Building block 3 can not dissolve in NMP. In the coupling step with 3, NMP-DMSO (3:4) was used to dissolve 3 in coupling solution.

After the sequences were synthesized, the N-terminal of each compound on the resin was acetylated with 3 equivalents of glacial acetic acid: BOP: HOBt: DIEA (1:1:1:1) with 10-min preactivation before coupling.

The acetylated resin was washed, dried in vacuo, and cleaved with 30%HFIP in DCM at room temperature (30 min). The DCM solution containing cleaved peptide was then dried by blowing nitrogen to leave a residue, which was dissolved in glacial acetic acid and lyophilized.

HPLC analysis showed that individual compound had one peak, indicating these 30 compounds are pure. ESI-MS measured molecular weight for each compound is consistent with the calculated value, confirmed the structure.

This synthesis demonstrated that using only 1.5 equivalents of amino acid, the coupling reaction was quite efficient. Both the yield and purity of product were high (>80%).
Synthesis of microcin B17 fragment 13-23 (39)

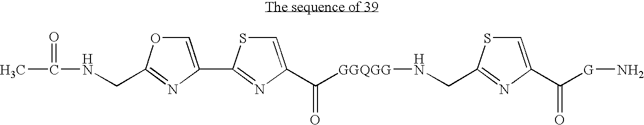

Microcin B17 fragment 13-23 (39) was synthesized on MBHA resin by Fmoc strategy. 2-Fmoc-aminomethyl-thiazole-4-carboxylic acid (1), 2-(2′-Fmoc-aminomethyl-oxazole-4′-yl)-thiazole-4-carboxylic acid (3), and Fmoc-glutamine (38) were synthesized in chapter 1. Coupling reaction was performed by two equivalents of Fmoc-amino acid: BOP: HOBt: DIEA (1:1:1:1) with 10-min preactivation before coupling (16).

MBHA resin was used to synthesize peptide amide. Fmoc-amino acid was coupled to the resin for 60 min. Coupling was monitored via a ninhydrin test.

Compound 3 can not dissolve in NMP. In the coupling step with 3, 1 ml of is DMSO was added in coupling solution.

After the sequence was synthesized, and Fmoc group was removed, the resin-bound peptide was acetylated by acetic anhydride (10 eq.) in the presence of DIEA in NMP for two hours and monitored by ninhydrin test.

Then, the resin was washed, dried in vacuo overnight, and cleaved with HF at 0° C. for 60 min without adding any scavenger.

HPLC analysis of the product showed that there was a main peak at 14.70 min (content >90%) with two minor peaks at 15.23 min (ca. 2%) and 15.66 min (ca. 5%) in the product.

UV spectrum of 39 showed two shoulder peaks at 223sh nm (ε 2.0×10⁴ M^{31 1} cm⁻¹) and 276sh nm (ε 8450). ESI-MS measured molecular weight of 39 is consistent with the calculated monoisotopic mass, confirmed the integrity of this peptide.

Experimental Section

Boc-D-3-(4thiazolyl) alanine and Boc-L-3-(4-thiazolyl) alanine were purchased from SyntheTech. 1,3-Diaminopropane trityl resin (0.83 mmol/g) and Fmoc-glycine were from Novabiochem. 1,1,1,3,3,3-Hexafluoro-2-propanol (HFIP) was from Aldrich. N, N′-Dicyclohexylcarbodiimide (DCC), N, N-diisopropylethylamine (DIEA), ninhydrin, 1-hydroxybenzotriazole (HOBt) and acetic anhydride were from Fluka. 4-Methylbenzhydrylamine resin (MBHA, 1.11 mmol/g, 200-400 mesh), trifluoroacetic acid (TFA), benzotriazole- 1 -yl-oxy-tris-(dimethylamino)-phosphonium hexafluorophosphate (BOP), and piperidine were from Advanced ChemTech. N-methylpyrrolidone (NMP), acetonitrile (HPLC grade, UV cutoff 189 nm), dimethyl sulfoxide (DMSO), and dichloromethane (DCM) were from Burdick & Jackson. 2-propanol (IPA) was from Fisher. Acetic acid glacial was from Mallinckrodt.

2-(Fmoc-aminomethyl)-thiazole-4-carboxylic acid (1), 2-(Fmoc-amino-methyl)-oxazole-4-carboxylic acid (2), 2-(2′-Fmoc-aminomethyloxazole-4′-yl)-thiazole-4-carboxylic acid (3), and Fmoc-glutamine (38) were synthesized in as previously discussed.

Ninhydrin Test

Reagent a: Mix solution 1 and solution 2 (solution 1: dissolve 40 g of phenol in 10 ml of absolute ethanol. Stir the solution with 4 g of Amberlite mixed-bed resin MB-3 for 45 min. Filter; solution 2: dissolve 33 mg of KCN in 50 ml of water. Dilute 2 ml of the KCN solution to 100 ml with pyridine. Stir with 4 g of Amberlite mixed-bed resin MB-3. Filter).

Reagent b: Dissolve 2.5 g of ninhydrin in 50 ml of absolute ethanol. Store in dark under nitrogen.

Procedures: A few beads of resin sample were removed into a test tube from reaction vessel using a glass pipette. The resin was washed with isopropanol by decantation. Four drops of reagent a and two drops of reagent b were added into the test tube and mixed well. The test tube was then placed in a preheated heating block at 100° C. for five min. Negative reaction was indicated by white beads and yellow solution through observing the test tube against a white background.

HPLC analyses were performed on a Waters HPLC system using a Vydac 218TP C18 10 μm reversed-phase column (250×4.6 mm). 18.2 MO water was produced by a Milli-Q plus system (Millipore, Bedford, Mass.).

UV spectra were recorded on a Hitachi U-2000 spectrophotometer. The molecular weight of products was determined by ESI-MS at Pfizer Central Research (Groton, Conn.) on a PE SCIEX API-100B LC/MS System (Foster City, Calif.). Mode: ESI, single quad, m/z=300-2200, 4.2 sec/scan, flow rate: 200 μl/min, acetonitrile-water (50:50) in 0.1% TFA (v/v). The data were processed using BioMultiview 1.3 alpha program.

The ¹H NMR spectrum was measured on a Briker 300 spectrometer, solvent: D₂O. Abbreviations for peak description are s=singlet, d=doublet, and m=multiplet.

Synthesis of Library One (L1)

This library was synthesized on MBHA resin by Boc strategy. Sixteen syringes of MULTIBLOCK simultaneous multiple peptide synthesizer were used for this library synthesis.

MBHA resin (40 mg, 0.0444 mmol) was placed in each syringe (10×45 mm). The resin in each syringe was washed with DCM (6×1.5 ml, 9 min), 10% DIEA in DCM (2×1.5 ml, 3 min), and DCM (6×1.5 ml, 9 min). According to the sequences in Table 3, Boc-D-3-(4-thiazolyl) alanine or Boc-L-3-(4-thiazolyl) alanine (48.3 mg, 0.1776 mmol, 4 eq.) was dissolved in 1 ml of DCM and added to the syringe (1.5 min). The syringes were shaken for 60 min after 178pw of IM DCC in DCM (36.7 mg, 0.1776 mmol) was added to each syringe. A resin sample was taken for ninhydrin test. The syringes were then washed with DCM (6×1.5 ml, 9 min). The Boc group was removed by shaking the syringe with 40% TFA in DCM (1×1 ml, 1.5 min; 1×1.5 ml, 30 min), and the deprotection was monitored by ninhydrin test.

Above cycle was repeated to continue the syntheses. After the sequences were synthesized, and Boc group was removed, the resin was washed with DCM (6×1.5 ml, 9 min) and dried in vacuum overnight. The dried resin was cleaved with HF at 0° C. for 60 min without adding any scavenger. After cleavage, the resin was extracted with 10% acetic acid aqueous solution (4×2 ml). The extraction solution was lyophilized to yield the peptide product. The results are depicted in Table 3.

TABLE 3
The data of the compounds in library one (L1).

| Compd | | Product | Yield | Retention time^{b} |
|---|---|---|---|---|
| No. | Structure^{a} | (mg) | (%) | (min) |
| 1 | DDDD-NH₂ | 9.9 | 35.2 | 8.73 |
| 2 | DDDL-NH₂ | 10.0 | 35.6 | 9.64 |
| 3 | LDDL-NH₂ | 7.7 | 27.4 | 10.28 |
| 4 | LDDD-NH₂ | 11.0 | 39.1 | 9.94 |
| 5 | DDLL-NH₂ | 8.7 | 31.0 | 9.06 |
| 6 | DDLD-NH₂ | 10.6 | 37.7 | 10.10 |
| 7 | LDLL-NH₂ | 15.1 | 53.7 | 10.92 |
| 8 | LDLD-NH₂ | 15.6 | 55.5 | 11.38 |
| 9 | DLLL-NH₂ | 14.3 | 50.9 | 9.94 |
| 10 | DLDL-NH₂ | 11.8 | 42.0 | 11.38 |
| 11 | LLDL-NH₂ | 14.2 | 50.5 | 10.10 |
| 12 | LLLL-NH₂ | 19.4 | 69.0 | 8.73 |
| 13 | DLDD-NH₂ | 18.3 | 65.1 | 10.92 |
| 14 | DLLD-NH₂ | 16.9 | 60.1 | 10.28 |
| 15 | LLLD-NH₂ | 17.5 | 62.3 | 9.64 |
| 16 | LLDD-NH₂ | 12.9 | 45.9 | 9.06 |

^{a}D: D-(3)-(4-thiazolyl) alanine, L: L-(3)-(4-thiazolyl) alanine.
^{b}HPLC system was described in General part. Mobile-phase gradient: 10%-30% acetonitrile in 0.1% (v/v) TFA over 20 min.; Flow rate 1.0 ml/min; UV detection: 215 nm; Sample injection: 5 μl. The retention time was reported in the average of two runs when the enantiomers were co-injected.
The spectroscopic data of L1-4 (LDDD-NH₂)
¹H-NMR(300MHz, D₂O)δ ppm: 8.88-8.87(4H, m), 7.29(1H, d, J=1.85Hz), 7.26(1H, d, J=1.82Hz), 7.22(1H, d, J=1.83Hz), 7.16(1H, d, J=1.82Hz), 4.67-4.57(4H, m), and 3.30-2.92(8H, m). ESI-MS(m/z): 634.1[M+1]⁺, calculated monoisotopic mass 634.11. UV: λₘₐₓ(H₂O)238nm(ε6500M⁻¹cm⁻¹).

Synthesis of Library Two (L2)

This library was synthesized on 1,3-diaminopropane trityl resin by Fmoc strategy. Fifteen syringes of a MTIBLOCK simultaneous multiple peptide synthesizer were used for this library synthesis. The syntheses of 2-Fmoc-anminomethyl-thiazole-4-carboxylic acid (1), 2-Fmoc-aminomethyl-oxazole-4-carboxylic acid (2), and 2-(2′-Fmoc-aminomethyloxazole-4′-yl)-thiazole-4-carboxylic acid (3) have been disclosed herein.

Coupling reaction was performed by 1.5 equivalents of Fmoc-amino acid: BOP: DIEA(1:1:1:1) with 10-min preactivation before coupling (16).

1,3-Diaminopropane trityl resin (0.83 mmol/g) (150 mg, 0.124 mmol) was placed syringe (10×45 mm). The resin in each syringe was washed with DCM (3×1.5 ml, 6 in), NMP (3×1.5 nil, 6 min), 5% DIEA in NMP (2×1.5 ml,3 min), and NMP (6×1.5 ml, 9 min).

According to the sequences in Table 2, Fmoc-amino acid (0.186 mmol, 1.5 eq.), BOP (82.3 mg, 0.186 mmol) and HOBt (25.7 mg, 0.186 mmol) were dissolved in 1 ml of NMP, followed by addition of DIEA (32.5 μl, 0.186 mmol). The solution was shaken for 10 min and added to the syringe. The syringes were shaken for 60 min. A resin sample was taken for ninhydrin test. The syringes were then washed with NMP (3×1.5 ml, 6 min), DCM-IPA (1:1) (3×1.5 ml, 6 min), IPA (3×1.5 min, 6 min), and NMP (3×1.5 ml, 9 min). The Fmoc group was removed by shaking the syringe with 20% piperidine in NMP (1×1.5 ml, 1.5 min; 1×1.5 ml, 20 min), and the deprotection was monitored by ninhydrin test. The syringes were then washed with NMP (3×1.5 ml, 6 min), DCM-IPA (1:1) 3×1.5 ml, 6 min), IPA (3×1.5 ml, 6 min), and NMP (3×1.5 ml, 9 min).

Above cycle was repeated to continue the syntheses. Compound 3 was dissolved in 1 ml of NMP-DMSO (3:4). In last cycle of acetylation, glacial acetic acid (21.3 1l, 0.372 mmol, 3 eq.), BOP (164.6 mg, 0.372 mmole), HOBt (51.4 mg, 0.372 mmole and DIEA (97.0 μl, 0.558 mmole, 4.5 eq.) were dissolved in 1 ml of NMP.

After the sequences were synthesized, the resin was washed with NMP (3×1.5 ml, 6 min), DCM-IPA (1:1) (3×1.5 ml, 6 min), IPA (3×1.5 ml, 6 min), and dried in vacuum overnight. The dried resin was cleaved with 30% HFIP in DCM at room temperature (2 ml, 30 min×3). After the peptide solution was dried by blowing N₂ , the residue was dissolved in 4 ml of glacial acetic acid. The acetic acid solution was lyophilized to yield the product. The results are depicted in Table 4.

TABLE 4
The data of the compounds in library two (L2).

| | | | UV^{b} λₘₐₓ | Retention | | |
|---|---|---|---|---|---|---|
| Compd | Product | Yield | (nm) | time | MW [M + 1]⁺ | |
| No.^{a} | (mg) | (%) | (ε × 10⁴) | (min)^{c} | Found^{d} | Calculated |
| 1 | 53.1 | 83.5 | 236 (1.67) | 5.45 | 454.2 | 454.13 |
| 2 | 51.6 | 83.5 | 214sh (1.90) | 4.21 | 438.2 | 438.16 |
| 3 | 57.0 | 79.2 | 276sh (1.10) | 12.46 | 521.5 | 521.14 |
| 4 | 50.5 | 82.0 | 214sh (1.93) | 4.58 | 438.2 | 438.16 |
| 5 | 47.1 | 79.0 | 210 (2.30) | 3.74 | 422.2 | 422.18 |
| 6 | 54.7 | 78.0 | 275 (0.82) | 10.44 | 505.2 | 505.16 |
| 7 | 54.9 | 72.2 | 276 (1.06) | 12.68 | 521.5 | 521.14 |
| 8 | 49.0 | 70.0 | 276 (0.60) | 9.08 | 505.2 | 505.16 |
| 9 | 62.2 | 77.3 | 276 (1.46) | 17.14 | 588.4 | 588.14 |
| 10 | 46.2 | 81.8 | 240 (1.67) | 7.35 | 397.3 | 397.11 |
| 11 | 46.2 | 84.6 | 210sh (1.87) | 5.02 | 381.3 | 381.13 |
| 12 | 48.8 | 89.4 | 214sh (1.73) | 5.25 | 381.3 | 381.13 |
| 13 | 45.5 | 86.5 | 214 (2.14) | 4.08 | 364.9 | 365.16 |
| 14 | 62.8 | 85.0 | 240 (3.07) | 15.91 | 537.1 | 537.12 |
| 15 | 60.5 | 89.0 | 213 (3.30) | 6.00 | 489.2 | 489.18 |

^{a}The structures see Table 2.
^{b}Solvent: water; uint of ε: M⁻¹ cm⁻¹; sh: shoulder peak.
^{c}HPLC system was described in General part. Mobile-phase gradient: 10%-30% acetonitrile in 0.1% (v/v) TFA over 20 min.; Flow rate 1.0 ml/min; UV detection: 214 nm and the λₘₐₓ; Sample injection: 5 μl or 2 μl (1 mM). The retention time was reported in the average of two runs.
^{d}The found molecular weight was determined by ESI-MS. The calculated one is the monoisotopic molecular weight.

Synthisis of microcin B17 fragment 13-23 (39)

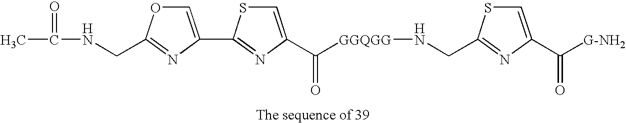

Microcin B17 fragment 13-23 (39) was synthesized on MBHA resin by Fmoc strategy. 2-Fmoc-aminomethyl-thiazole-4-carboxylic acid (1), 2-(2′-Fmoc-aminomethyl-oxazole-4′-yl)-thiazole-4-carboxylic acid (3), and Fmoc-glutamine (38) were synthesized as previously discussed. Coupling reaction was performed by two equivalents of Fmoc-amino acid: BOP: HOBt: DIEA (1:1:1:1) with 10-min preactivation before coupling.

MBHA resin (0.10 g, 0.111 mmol) was placed in a syringe reaction vessel (10×45 mm). The resin was washed with NMP (3×1.5 ml, 6 min), DCM-IPA (1:1) (3×1.5 ml, 6 min), IPA (3×1.5 ml, 9 min), and NMP (3×1.5 ml, 9 min).

According to the sequence of 39, Fmoc-amino acid (0.222 mmole, 2 eq.) was dissolved in 444 μl of 0.5 M BOP solution in NMP and 444 μl of 0.5 M HOBt solution in NMP by vortexing. After 444 μl of 0.5 M DIEA solution in NMP was added to above solution and shaken for 10 min, the solution was added to the reaction vessel.

The reaction vessel was shaken for 60 min. A resin sample was taken for ninhydrin test. The reaction vessel was then washed with NMP (3×1.5 ml, 6 min), DCM-IPA (1:1) (3×1.5 ml, 6 min), IPA (3×1.5 ml, 9 min) and NMP (6×1.5 ml, 9 min). The Fmoc group was removed by shaking the vessel with 20% piperidine in NMP (1×1.5 ml, 1.5 min; 1×1.5 ml, 20 min), and the deprotection was monitored by ninhydrin test.

Above cycle was repeated to continue the synthesis. Compound 3 was dissolved in 1 ml of DMSO, followed by addition of 444 μl of 0.5 M BOP solution in NMP, 444 μl of 0.5 M HOBt solution in NMP, and 39 μl of DIEA.

After the sequence was synthesized, and Fmoc group was removed, the resin-bound peptide was acetylated by shaking reaction vessel with a solution of acetic anhydride (105 μl, 1.11 mmol, 10 eq.) and DIEA (193 μl, 1.11 mmole) in 1.5 ml of NMP for two hours and monitored by ninhydrin test. The resin was washed with NMP (3×1.5 ml, 6 min), DCM-IPA (1:1) (3×1.5 ml, 6 min), IPA (6×1.5 ml, 9 min) and dried in vacuum overnight. The dried resin was cleaved with HF at 0° C. for 60 min without adding any scavenger. After cleavage, the resin was extracted with glacial acetic acid (4×2 ml). The extraction solution was lyophilized to yield the peptide product (23.8 mg, yield 26%).

HPLC analysis Mobile-phase gradient: 10%-45% acetonitrile in 0.1% (v/v) over 35 min; Flow rate: 1 ml/min; UV detection: 215 and 276 nm; Sample: 5 μl (1 mM) was injected; The retention time of main peak: 14.70 min. (purity >90%).

UVλ (H₂O) (nm): 223sh (ε 2.0×10⁴ M⁻¹ cm⁻¹) and 276sh (ε 8450).

ESI-MS (m/z): 820.3 [M+1]⁺, calculated monoisotopic molecular weight 820.23.

Bioassay of the Peptidomimetic Libraries

The thiazole and oxazole-containing peptides from natural sources have important biological activities such as antitumor, antifungal, antibiotic and antiviral activities. To establish if the thiazole and oxazole ring systems could be important pharmacophores in those biologically active peptides, two libraries of thiazole and/or oxazole-containing peptidomimetics, and a microcin B17 fragment 39 were synthesized and found to have antibiotic activity including antibacterial and antifungal.

The DNA binding activity of the tetrapeptide amides in the first library was measured using capillary zone electrophoresis. The results are depicted in Table 5.

TABLE 5
Binding Constants of the 15 peptide amides in Library 1

| Com- | | | | | |
|---|---|---|---|---|---|
| pound | Sequence | Kₐ(1)^{b} | Compd. | Sequence | Kₐ(1)^{b} |
| 12 | LLLL-NH₂ | 2.1 × 10⁶ | 3 | LDDL-NH₂ | 2.0 × 10⁴ |
| 9 | DLLL-NH₂ | 4.2 × 10⁵ | 14 | DLLD-NH₂ | 1.8 × 10⁴ |
| 11 | LLDL-NH₂ | 1.9 × 10⁵ | 10 | DLDL-NH₂ | 1.5 × 10⁴ |
| 8 | LDLD-NH₂ | 5.5 × 10⁴ | 13 | DLDD-NH₂ | 1.4 × 10⁴ |
| 15 | LLLD-NH₂ | 5.2 × 10⁴ | 16 | LLDD-NH₂ | 1.4 × 10⁴ |
| 7 | LDLL-NH₂ | 2.8 × 10⁴ | 1 | DDDD-NH₂ | 2.5 × 10⁴ |
| 2 | DDDL-NH₂ | 2.4 × 10⁴ | 5 | DDLL-NH₂ | 1.7 × 10⁴ |
| 6 | DDLD-NH₂ | 2.3 × 10⁴ | | | |

Peptides are listed in the order of Kₐ(1) value from highest to lower value.
^{b}Kₐ(1) (M⁻¹ is the stoichiometric equilibrium binding constant near saturation.

In addition, peptides L1-3, L1-5, L1-7, L1-13, L1-14, and L1-16 were evaluated for inhibition of the growth of rat hepatoma cell lines 1682A, 1682B, 1683.1.4 and T252. No growth inhibition was observed in both 10% serum and serum-free media.

The method used to determine the cell growth inhibition activity of the compounds in the second library and also a fragment of microcin B 17, compound 39 is herein described.

Results and Discussion

The marin bacterium Vibrio anguillarum, a fish pathogen causing the disease “vibriosis” in marine fish and shellfish were used in this experiment.

Bacteria (V. anguillarum) were grown overnight at 28° C. in Luria-Bertani (LB) 20 medium (the concentration of NaCl is 20 g/L for marine bacteria in LB medium, instead of the usual 10 g/L). The culture was re-inoculated and incubated in LB20 medium at 28° C. (ca. 2hr) to reach the exponential growth phase of the bacteria. The bacterial suspension was then dilute with 0.1×LB20 medium to make the bacterial dilution containing 2×10³ colony-forming units (cfm)/ml.

At time zero, the bacteria were treated with the peptide in 0.1×LB20 medium. After incubation for 3 hours, 1×LB20 medium was added to the culture, and incubated for 20 hours. The results showed that peptides L2-6, L2-9 and 39 inhibited the growth of bacterial cultures. However, these peptides did not kill the bacteria, because the increase in the optical density (OD) of the cultures at different incubation times showed the cells were still growing in the cultures.

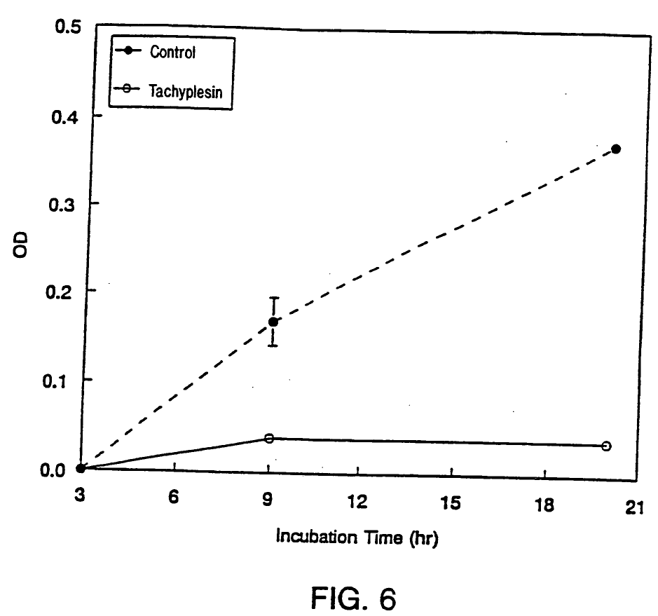

The antibiotic peptide tachyplesin was used to assess the bacterial assay used. Compared to tachyplesin (FIG. 6 and Table 8), the bacteria treated with peptides L2, L2-9 and 39 gradually recovered their ability to divide.

The effect of peptides L2-6, L2-9 and 39 on the growth of V. anguillarum is very similar to the effect of microcin B 17 on the growth of cells which are immune to microcin B17.

A structural comparison of peptides L2-6, L2-9 and microcin B 17 fragment 39 revealed that they have the identical N-terminal moiety, the acetyl-oxazolyl thiazole amino acid building block. Further comparison of these structures with other peptides in the library indicated that the N-terminal moiety must not be the only requirement for the activity, because peptide L2-3 has the same N-terminal moiety and L2-3 did not have a detectable effect on the growth of V. anguillarum.

TABLE 6
OD₆₅₀^{a} of V. anguillarum cultures after incubation with peptides
L2-6 and L2-9 for 9 and 20 hr at 28°

| Compund | L2-6 | | L2-9 | | Growth Control | |
|---|---|---|---|---|---|---|
| Incubation time (hr) | 9 | 20 | 9 | 20 | 9 | 20 |
| No peptide added | | | | | 0.138 | 0.400 |
| Dilution 1 (500 μM) | 0.073 | 0.388 | 0.075 | 0.354 | | |
| Dilution 2 (250 μM) | 0.113 | 0.427 | 0.125 | 0.409 | | |
| Dilution 3 (125 μM) | 0.120 | 0.402 | 0.131 | 0.425 | | |
| Dilution 4 (62.5 μM) | 0.130 | 0.391 | 0.135 | 0.421 | | |
| Dilution 5 (31.2 μM) | 0.137 | 0.395 | 0.135 | 0.417 | | |

^{a}OD₆₅₀ is the average of measurements from three different wells.

TABLE 7
OD₆₅₀^{a} of V. anguillarum cultures after incubation with microcin
B17 fragment 39 for 9 and 20 hr at 28°

| | | |
|---|---|---|
| Compound | 39 | Growth control |

| Incubation time (hr) | 9 | 20 | 9 | 20 |
|---|---|---|---|---|
| No peptide added | | | 0.126 | 0.407 |
| Dilution 1 (500 μM) | 0.034 | 0.244 | | |
| Dilution 2 (250 μM) | 0.034 | 0.241 | | |
| Dilution 3 (125 μM) | 0.051 | 0.279 | | |
| Dilution 4 (62.5 μM) | 0.082 | 0.297 | | |
| Dilution 5 (31.2 μM) | 0.111 | 0.419 | | |

^{a}OD₆₅₀ is the average of measurements from three different wells.

TABLE 8
OD₆₅₀^{a} of V. anguillarum cultures containing the antibacterial peptide
tachyplesin after incubation for 9 and 20 hr at 28°

| | | |
|---|---|---|
| Compound | tachyplesin | Growth control |

| Incubation time (hr) | 9 | 20 | 9 | 20 |
|---|---|---|---|---|
| No peptide added | | | 0.170 | 0.374 |
| Dilution 1 (50 μg/ml) | 0.039 | 0.038 | | |
| Dilution 2 (25 μg/ml) | 0.035 | 0.035 | | |
| Dilution 3 (12.5 μg/ml) | 0.035 | 0.035 | | |
| Dilution 4 (6.25 μg/ml) | 0.068 | 0.036 | | |
| Dilution 5 (31.2 μg/ml) | 0.098 | 0.034 | | |

^{a}OD₆₅₀ is the average of measurements from three different wells.

All journal articles and reference citations provided above, in parentheses or otherwise, whether previously stated or not, are incorporated herein by reference.

The foregoing description has been limited to a specific embodiment of the invention. It will be apparent, however, that variations and modifications can be made to the invention, with the attainment of some or all of the advantages of the invention. Therefore, it is the object of the appended claims to cover all such variations and modifications as come within the true spirit and scope of the invention.

Having described our invention, what we now claim is:

1. (canceled)

2. (canceled)

3. An method for producing a N-protected oxazole and thiazole amino acid comprising the structure of: 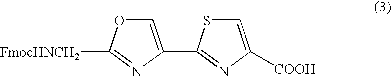
which comprises:
removing the Fmoc protective group of TrOCH₂ FmocNH-CH-COOH
to produce 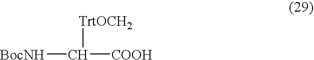
effecting a reaction with (29) to produce 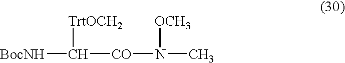
reducing (30) to produce 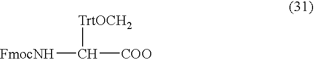
condensing (31) to produce 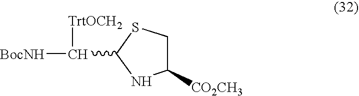
dehydrogenating (32) to produce 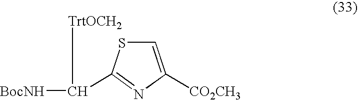
removing the Boc and Trt protecting groups to produce 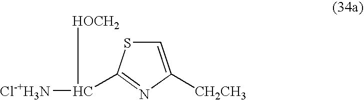
effecting a reaction with (34a) to produce 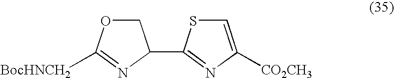
dehydrogenating (35) to produce 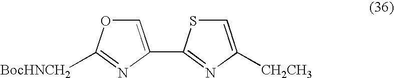 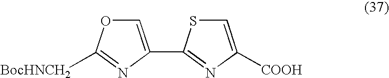
converting the Boc protective group of (37) to a Fmoc protecting group to produce (3).

4. A N-protected oxazole and thiazole amino acid comprising the structure of:

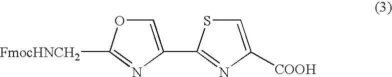

5. A combinatorial library, of at least two compounds, each compound within the library being derived from the solid phase peptide combinatorial synthesis of at least one compound selected from the group consisting of: 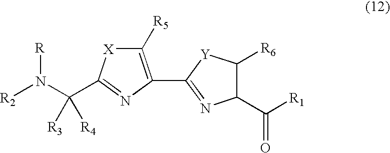
where R and R₂═H, a naturally occurring or synthetic L or D amino acid, Teributyloxycarbonyl (Boc), 9-fluorenyttnethoxycarbonyl (Fmoc), carbobenzoxy (Z), Benzozyl (Bz), and other like amino protecting groups;
where R₁═OH, alkyl esters, aromatic esters such as methyl, ethyl, t-butyl and benzyl, a naturally occurring or synthetic L or D amino acid, activated esters such as pentafluorophenyl, nitrophenyl, N-hydroxysuccinimide, acid chlorides, fluorides, organic salts, such as cyclohexyamines (CHA), amides, an amide bonded to a linker such as a diamine, or an insoluble support for use in solid phase synthesis;
where R₃₋₄═C₁-C₁₀ alkyl, a heterocylic ring, an aliphatic or aromatic ring, a functional group such as an amine, an alchohol, a halide or an organometallic complex
where R₅₋₆═H,
where X═oxygen (O) or sulfur (S);
where Y═oxygen (O) or sulfur (S);
wherein at least one of the compounds selected from the group consisting 11 and 12 forms an amide bond with at least one of the compounds selected from the group consisting of 11 and 12 or a naturally occurring or synthetic amino acid.

6. (canceled)

7. (canceled)

8. (canceled)
